(19) United States
(12) Patent Application Publication
(10) Pub. No.: US 2011/0311621 A1
(43) Pub. Date: Dec. 22, 2011

(54) PHARMACEUTICAL COMPOSITIONS AND METHODS OF DELVERY

(76) Inventors: Paul Salama, Ashdod (IL); Irina Karmeli, Maale Adummim (IL); Shmuel Tuvia, Netanya (IL); Karen Marom, Mevasseret Zion (IL); Oshrat Harush-Frenkel, Modi'in (IL); Dana Gelbaum, Tel Aviv (IL); Roni Mamluk, Mazkeret Batia (IL); Fredric David Price, Bedford, NY (US); Steven Ronald Peikin, Philadelphia, PA (US)

(21) Appl. No.: 13/049,681

(22) Filed: Mar. 16, 2011

Related U.S. Application Data
(60) Provisional application No. 61/314,430, filed on Mar. 16, 2010.
(60) Provisional application No. 61/436,781, filed on Jan. 27, 2011.

(51) Int. Cl. A61K 9/48; A61P 3/04; A61K 38/08; A61P 3/00; A61K 9/00; A61K 9/28

(52) U.S. Cl. 424/463; 424/400; 424/474; 514/21.7

(57) ABSTRACT

The pharmaceutical compositions described herein include a suspension which comprises an admixture in solid form of a therapeutically effective amount of a therapeutic agent (such as CCK-8, octreotide), at least one salt of a medium chain fatty acid, a matrix forming polymer and a hydrophobic(lipophilic) medium. A surfactant may be included in the suspension. The pharmaceutical compositions may be formulated in a capsule or tablet for oral delivery. Methods of treating or preventing diseases by administering such compositions to affected subjects are also disclosed.

### RELATED APPLICATIONS

This application claims benefit under 35 U.S.C.s. 119(e) to U.S. Provisional Application Ser. Nos. 61/314,430, entitled “PHARMACEUTICAL COMPOSITIONS AND METHODS OF DELIVERY,” filed Mar. 16, 2010, and 61/436,781, entitled “PHARMACEUTICAL COMPOSITIONS AND METHODS OF DELIVERY,” filed Jan. 27, 2011, which are each incorporated by reference herein in their entirety.

### FIELD OF THE TECHNOLOGY

The present invention relates generally to pharmaceutical compositions enabling improved delivery e.g., oral delivery and methods of using such compositions.

### BACKGROUND

Techniques enabling efficient transfer of a substance of interest across a biological barrier are of considerable interest in the fields of biotechnology and medicine. For example, such techniques may be used for the transport of a variety of different substances across a biological barrier regulated by tight junctions (i.e., the mucosal epithelia, which include the intestinal and respiratory epithelia, and the vascular endothelia, which include the blood-brain barrier, nasal membrane, cornea and other eye membranes, and genito-urinary membranes). In particular there is great interest in oral delivery of therapeutic agents to avoid the use of more invasive means of administration and hence improve patient convenience and compliance. Administration of such a substance via the oral route is preferred to parenteral administration because it allows self-administration by patients whereas parenteral formulations have to be administered in most cases by a physician or paramedical personnel.

Diverse drug delivery vehicles have been employed, among them liposomes, lipidic or polymeric nanoparticles, and microemulsions. These have improved the oral bioavailability of certain drugs, mostly by the protective effect they offer. However, for most relevant drugs, bioavailability remains very low and fails to achieve the minimal therapeutic goals. Hence, a need exists for an efficient, specific, non-invasive, low-risk means to target various biological barriers for the non invasive delivery of various therapeutic agents such as peptides and polypeptides, macromolecule drugs and other therapeutic agents which include small molecules with low bioavailability.

### SUMMARY

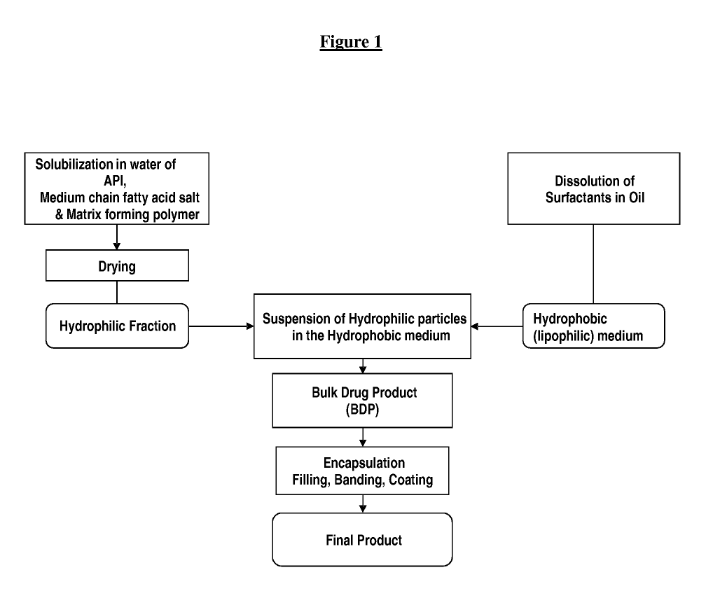

Described herein are novel pharmaceutical compositions, processes for making these compositions, and methods of treating subjects using these compositions. The present inventors have devised a process for producing a pharmaceutical composition (bulk drug product) which involves preparing a water soluble composition comprising a therapeutically effective amount of at least one therapeutic agent (API), a medium chain fatty acid salt and a matrix forming polymer, drying (e.g., by lyophilization) the water soluble composition to obtain a solid powder, and suspending the lyophilized material (the solid powder) in a hydrophobic(lipophilic) medium, preferably castor oil or glyceryl tricaprylate (including other ingredients e.g., surfactants (viscosity modifiers)—see below, to produce an oily suspension containing in solid form the therapeutic agent, the medium chain fatty acid salt and the matrix forming polymer, thereby producing the bulk drug product. The solid form may comprise a particle (e.g., consists essentially of particles, or consists of particles). The particle may be produced by lyophilization or by granulation or by spray-drying or by other means. The bulk drug product may then be encapsulated in capsules (or formed into tablets) which may be coated by a pH sensitive coating and may be used for oral delivery. A typical generic process for producing the claimed formulations is shown in FIG. 1.

The inventors of the present invention have discovered that the absorption of certain therapeutic agents in a subject can be improved when administered in the compositions described herein. For example, a therapeutic agent administered in a formulation in accordance with one or more embodiments exhibits an improved bioavailability (BA) relative to the same therapeutic agent administered via a similar route but in a composition substantially free of the components described herein or having a lower amount of the components described herein; such components are e.g., medium chain fatty acid salt, a matrix forming polymer, a hydrophobic medium. Such improvement in relative BA may be on the order of at least about 1.5-, 2-, 3-, 5-, 10-, 50- or 100-fold. In some aspects, a composition described herein enables the absorption in the gastrointestinal (GI) tract of a therapeutic agent that is generally characterized by low or zero oral bioavailability and/or absorption. These therapeutic agents may have low or zero bioavailability, e.g., in aqueous solution, and in other oral formulations known in the art. In at least one aspect, a composition described herein improves bioavailability by enhancing the GI wall/barrier permeability to the drug molecules. For example, a composition described herein may enhance absorption by permeating the GI wall/barrier via unsealing of the tight junctions between GI epithelial cells, allowing paracellular absorption in addition to the existing transcellular absorption.

The present invention demonstrates delivery of the product to the intestine, which is a model for oral delivery, and from there to the bloodstream with high bioavailability.

One aspect of the present invention demonstrates a novel formulation for cholecystokinin-8 (CCK-8). Another aspect of the present invention demonstrates the oral administration of CCK-8. The oral administration of CCK-8 may be by means of an enteric coated oral dosage form, in particular a capsule or a tablet. Another aspect of the present invention demonstrates a method of treatment of an obese or overweight subject comprising orally administering to the patient, in an enteric coated oral dosage form, a therapeutically effective amount of CCK-8 sufficient to produce weight loss. This method of treatment of an obese or overweight subject may be prior to bariatric surgery.

Still other aspects, embodiments, and advantages of these exemplary aspects and embodiments, are discussed in detail below. Moreover, it is to be understood that both the foregoing information and the following detailed description are merely illustrative examples of various aspects and embodiments, and are intended to provide an overview or framework for understanding the nature and character of the claimed aspects and embodiments. The accompanying drawing is included to provide illustration and a further understanding of the various aspects and embodiments, and is incorporated in and constitutes a part of this specification. The drawing, together with the remainder of the specification, serves to explain principles and operations of the described and claimed aspects and embodiments.

Throughout this application, various publications, including United States patents, are referenced by author and year and patents and applications by number. The disclosures of these publications and patents and patent applications in their entireties are hereby incorporated by reference into this application in order to more fully describe the state of the art to which this invention pertains.

### BRIEF DESCRIPTION OF THE DRAWINGS

Various aspects of at least one embodiment are discussed below with reference to the accompanying FIG. 1. The Figure is provided for the purposes of illustration and explanation and is not intended as a definition of the limits of the invention. FIG. 1 presents a generic process for production of a formulation of a composition of the invention in accordance with one or more embodiments as referenced in the accompanying Examples.

### DETAILED DESCRIPTION

Pharmaceutical compositions: The pharmaceutical compositions described herein include a therapeutic agent, a medium chain fatty acid salt and a matrix forming polymer in intimate contact or association with a substantially hydrophobic(lipophilic) medium. For example, the therapeutic agent and the medium chain fatty acid or derivative thereof, are in a solid form within the hydrophobic medium forming a suspension. They may be coated, suspended, sprayed by or immersed in the hydrophobic medium. The compositions of the invention are not emulsions. Almost all of the compositions are oily suspensions and the amount of water in the compositions is very low. A few of the present compositions (e.g., of aliskiren) incorporate a high amount (about 60-80% octanoic acid) which is a suspension at the concentration of solids exemplified, but at a lower concentration of solids (below the saturation threshold) a solution is obtained. The suspension may be a liquid suspension incorporating solid material, or a semi-solid suspension incorporating solid material (an ointment).

Many of the compositions described herein comprise a suspension which comprises an admixture of a hydrophobic medium and a solid form wherein the solid form comprises a therapeutically effective amount of a therapeutic agent and at least one salt of a medium chain fatty acid, and wherein the medium chain fatty acid salt is preferably present in the composition at an amount of 10% or more by weight, and a matrix forming polymer and/or a sugar. The solid form may comprise a particle (e.g., consist essentially of particles, or consist of particles). The particle may be produced by lyophilization or by granulation or by spray-drying or by other means. In some embodiments, preferably after milling, about 10% (v/v) of the particles are above about 120-140 microns, and about 50% (v/v) of the particles are above about 40-50 microns.

A cargo compound is a therapeutic agent (e.g., CCK-8, octreotide or aliskiren) or a test compound (e.g., high molecular weight dextran) which is formulated as described herein within the compositions of the invention.

In some preferred embodiments, compositions of the invention include only excipients which are generally recognized as safe, based on available data on human use, animal safety and regulatory guidelines (e.g., GRAS excipients). Some compositions of the invention may have other types of excipients (e.g., non-GRAS). In some embodiments the compositions of the invention have amounts of excipients that are within the maximum daily doses as noted in such available data for each specific excipient.

The medium chain fatty acid salt may generally facilitate or enhance permeability and/or absorption of the therapeutic agent. The matrix forming polymer (see below) serves to increase the effect of the permeability enhancer. In some embodiments the medium chain fatty acid salts include derivatives of medium chain fatty acid salts. The therapeutic agent, the medium chain fatty acid salt and the matrix forming polymer are in solid form, for example, a solid particle such as a lyophilized particle, granulated particle, pellet or micro-sphere. In preferred embodiments, the therapeutic agent, the medium chain fatty acid salt and the matrix forming polymer are all in the same solid form, e.g., all in the same particle. In other embodiments, the therapeutic agent, the medium chain fatty acid salt and the matrix forming polymer may each be in a different solid form, e.g., each in a distinct particle.

Unlike emulsions, where water is an essential constituent of the formulation, the compositions described herein provide a solid form such as a particle containing the therapeutic agent, which is then associated with the hydrophobic (oily) medium. The amount of water in the compositions is generally less than 3% by weight, usually less than about 2% or about 1% or less by weight.

The compositions described herein are suspensions which comprise an admixture of a hydrophobic(lipophilic) medium and a solid form wherein the solid form comprises a therapeutically effective amount of a therapeutic agent, at least one salt of a medium chain fatty acid and a matrix forming polymer. The solid form may be a particle (e.g., consist essentially of particles, or consist of particles). The particle may be produced by lyophilization or by granulation or by spray-drying or by other means. The medium chain fatty acid salt is generally present in the compositions described herein at an amount of 10% or more by weight. In certain embodiments the medium chain fatty acid salt is present in the composition at an amount of 10%-50%, or at an amount of about 10% -20% or about 10-15% or about 15-20%, preferably 11%-18% or about 11%-17% or 12%-16% or 12%-15% or 13%-16% or 13%-15% or 14%-16% or 14%-15% or 15%-16% or most preferably 15% or 16% by weight, and the medium chain fatty acid has a chain length from about 6 to about 14 carbon atoms preferably 8, 9 or 10 carbon atoms.

In some embodiments in the compositions described above, the solid form including the therapeutic agent also includes a stabilizer (to prevent or reduce degradation e.g., chemical degradation of the formulation upon storage) and includes stabilizers of protein structure which are compounds that stabilize protein structure under aqueous or non-aqueous conditions or can reduce or prevent aggregation of the therapeutic agent, for example during a drying process such as lyophilization or other processing step. A stabilizer can be for example, a polyanionic molecule, a polyvalent ion, a saccharide, a sugar alcohol, an amino acid, a polycationic molecule or other suitable compound, or a combination thereof.

The inventors unexpectedly found that, in some embodiments of the invention described herein, PVP, in particular PVP-12, serves to increase the effect of the permeability enhancer in a synergistic manner; furthermore, increasing the level of PVP-12 to 10% increased the absorption of the therapeutic agent into the blood due to the improved bioavailability of the formulations.

In certain particular embodiments, the matrix forming polymer is polyvinylpyrrolidone (PVP), and the polyvinylpyrrolidone is present in the composition at an amount of about 2% to about 20% by weight, preferably at an amount of about 3% to about 18% by weight, more preferably at an amount of about 5% to about 15% by weight, most preferably at an amount of about 10% by weight. In certain particular embodiments the polyvinylpyrrolidone is PVP-12 and/or has a molecular weight of about 3000.

The inventors demonstrated that dextran had a similar (but lower) effect as PVP did. Other matrix forming polymers were found to have a similar effect. Instead of PVP in the formulation, a range of matrix forming polymers were substituted e.g., carbomers (Carbopol® polymers) or alginate or hyaluronate or polyacrylic acid sodium salt; glucosamine or glucose was also substituted (see Tables 1A and 1B). All formulations showed bioavailability. The matrix forming polymers which produced a higher or similar bioavailability in the formulations of the invention as PVP were Carbopol polymer and PVA (polyvinyl alcohol); glucose also gave similar results to PVP. Carbopol polymers are polymers of acrylic acid cross-linked e.g., with polyalkenyl ethers or divinyl glycol. Carbopol 934P gave particularly high bioavailability. Carbopol® 934P is a high molecular weight polymer of acrylic acid crosslinked with allyl ethers of sucrose. PVA is a water-soluble synthetic polymer of vinyl alcohol monomers.

Note that replacing PVP-12 in the formulation, by e.g., Carbopol 934P or by PVA or by some of the other matrix forming polymers indicated, reduces the total amount of matrix forming polymer in the particle phase (i.e. the solid form) of the formulation (the hydrophilic fraction) and thus bestows the ability to load more API into the formulation, which may be desirable in order to achieve desired blood levels or reduce capsule size and number.

The amount of solid form (i.e. hydrophilic fraction) in the formulations of the invention is normally from about 10% to about 50% of the formulation (w/w). In certain aspects of the invention, the amount of solid form is from about 17% to about 40%.

In some embodiments, such as when the therapeutic agent is a small molecule, a bulking agent may be added.

In certain embodiments of the compositions described herein the therapeutic agent is a protein, a polypeptide, a peptide, a glycosaminoglycan, a small molecule, a polysaccharide or a polynucleotide.

In a particular embodiment of the compositions described herein the salt of the fatty acid is sodium octanoate and the hydrophobic medium is glyceryl tricaprylate or castor oil; in another particular embodiment the composition further comprises glyceryl monooleate and sorbitan monopalmitate or glyceryl monocaprylate and glyceryl tricaprylate and polyoxyethylenesorbitan monooleate; in another particular embodiment the composition further comprises glyceryl tributyrate or lecithin or ethylisovalerate or a combination thereof and at least one stabilizer; in another particular embodiment the composition further comprises a bile salt. Examples of bile salts are sodium taurocholate, sodium deoxycholate, sodium glycocholate, sodium chenodeoxycolate, sodium cholate, sodium lithocholate, in particular sodium taurocholate. In particular embodiments the therapeutic agent is insulin, growth hormone, parathyroid hormone or analogs thereof e.g., parathyroid hormone amino acids 1-34 termed teriparatide, interferon-alfa (IFN-α), a low molecular weight heparin, leuprolide, fondaparinux, octreotide, exenatide, terlipressin, vancomycin, gentamicin, cholecytokinin or analogs thereof, cholecytokinin-8 (CCK-8) or analogs thereof, calcitonin or aliskiren or salts of these therapeutic agents.

### Therapeutic Agents:

The pharmaceutical compositions described herein can be used with a variety of therapeutic agents (also termed active pharmaceutical ingredient=API). In some embodiments, the pharmaceutical composition includes a plurality of therapeutic agents (effectors). The therapeutic agents can either be in the same solid form (e.g., in the same particle), or the therapeutic agents can each be in an independent solid form (e.g., each in different particles). In certain embodiments, the therapeutic agent is in the form of a particle, for example, a granulated or solid particle. The particle is associated with or is in intimate contact with a substantially hydrophobic medium, for example, a hydrophobic medium described herein.

Therapeutic agents that can be used in the compositions described herein include any molecule or compound serving as, for example, a biological, therapeutic, pharmaceutical, or diagnostic agent including an imaging agent. The therapeutic agents include drugs and other agents including, but not limited to, those listed in the United States Pharmacopeia and in other known pharmacopeias. Therapeutic agents are incorporated into the formulations of the invention without any chemical modification. Therapeutic agents include proteins, polypeptides, peptides, polynucleotides, polysaccharides and small molecules.

The term “small molecule” is understood to refer to a low molecular weight organic compound which may be synthetically produced or obtained from natural sources and typically has a molecular weight of less than 2000 Da, or less than 1000 Da or even less than 600 Da e.g., less than or about 550 Da or less than or about 500 Da or less than or about 400 Da; or about 400 Da to about 2000 Da; or about 400 Da to about 1700 Da. Examples of small molecules are ergotamine (molecular weight=582 Da), fondaparinux (molecular weight=1727 Da), vancomycin (molecular weight=1449 Da), gentamicin (molecular weight=478 Da), doxorubicin (molecular weight=544 Da), aliskiren free base (molecular weight=552 Da) and aliskiren hemi-fumarate (molecular weight=610 Da).

The term “polynucleotide” refers to any molecule composed of DNA nucleotides, RNA nucleotides or a combination of both types which comprises two or more of the bases guanidine, citosine, timidine, adenine, uracil or inosine, inter alia. A polynucleotide may include natural nucleotides, chemically modified nucleotides and synthetic nucleotides, or chemical analogs thereof and may be single-stranded or double-stranded. The term includes “oligonucleotides” and encompasses “nucleic acids”.

By “small interfering RNA” (siRNA) is meant an RNA molecule (ribonucleotide) which decreases or silences (prevents) the expression of a gene/ mRNA of its endogenous or cellular counterpart. The term is understood to encompass “RNA interference” (RNAi), and “double-stranded RNA” (dsRNA). siRNA molecules are normally double-stranded RNA molecules 17-27 nucleotides in length.

By “polypeptide” is meant a molecule composed of covalently linked amino acids and the term includes peptides, polypeptides, proteins and peptidomimetics. A peptidomimetic is a compound containing non-peptidic structural elements that is capable of mimicking the biological action(s) of a natural parent polypeptide. Some of the classical peptide characteristics such as enzymatically scissile peptidic bonds are normally not present in a peptidomimetic.

The term “amino acid” refers to a molecule which consists of any one of the 20 naturally occurring amino acids, and also amino acids which have been chemically modified and synthetic amino acids.

By “polysaccharide” is meant a linear or branched polymer composed of covalently linked monosaccharides; glucose is the most common monosaccharide and there are normally at least eight monosaccharide units in a polysaccharide and usually many more.

Polysaccharides have a general formula of Cx(H2O)y where x is usually a large number between 200 and 2500. Considering that the repeating units in the polymer backbone are often six-carbon monosaccharides, the general formula can also be represented as (C₆H₁₀O₅)ₙ where 40≦n≦3000 i.e. there are normally between 40 and 3000 monosaccharide units in a polysaccharide.

A “glycosaminoglycan” is a polysaccharide that contains amino containing sugars. Exemplary anionic therapeutic agents include polynucleotides from various origins, and particularly from human, viral, animal, eukaryotic or prokaryotic, plant, or synthetic origin, etc including systems for therapeutic gene delivery. A polynucleotide of interest may be of a variety of sizes, ranging from, for example, a simple trace nucleotide to a gene fragment, or an entire gene. It may be a viral gene or a plasmid. Exemplary polynucleotides serving as therapeutic agents include specific DNA sequences (e.g., coding genes), specific RNA sequences (e.g., RNA aptamers, antisense RNA, short interfering RNA (siRNA) or a specific inhibitory RNA (RNAi)), poly CPG, or poly I:C synthetic polymers of polynucleotides.

Alternatively, the therapeutic agent can be a protein, such as, for example, an enzyme, a hormone, an incretin, a proteoglycan, a ribozyme, a cytokine, a peptide, an apolipoprotein, a growth factor, a bioactive molecule, an antigen, or an antibody or fragment(s) thereof such as a single chain antibody, etc. The peptide can be a small peptide e.g., from about 2 to about 40 amino acids.

Other examples of therapeutic agents include, but are not limited to hormones such as insulin. Other examples of therapeutic agents include, but are not limited to antibiotics, analgesic agents, anti-migraine agents, anti-coagulant agents, anti-emetic agents, cardiovascular, anti-hypertensive and vasodilator agents, sedatives, narcotic antagonists, chelating agents, anti-diuretic agents and anti-neoplastic agents. Particular embodiments of the therapeutic agent are insulin, growth hormone, parathyroid hormone or analogs thereof such as teriparatide, interferon-alfa (IFN-α), a low molecular weight heparin, leuprolide, fondaparinux, octreotide, exenatide, terlipres sin, vancomycin, gentamicin, cholecystokinin or analogs thereof such as cholecystokinin -8, calcitonin or aliskiren, or salts thereof.

The therapeutic agent can itself be directly active or can be activated in situ by the composition, by a distinct substance, or by environmental conditions. In some embodiments, the composition can include a plurality of therapeutic agents (combination drugs).

In some embodiments, the composition can include a small molecule and a peptide or protein. Combinations of two small molecules can be used when one of them generally has poor absorption or bioavailability even if the other generally has effective absorption or bioavailability, such as some antibiotics. Thus the compositions of the invention can include a second therapeutic agent. Compositions of the invention which include a third therapeutic agent are also envisaged.

In some embodiments of the compositions described herein, the composition includes a combination of a protein or peptide with small molecules that either do or do not have good absorption or bioavailability. For example, a composition can include at least one therapeutic agent that may generally be characterized as poorly absorbable or poorly bioavailable. The composition can also be used for the administration of therapeutic agents that are absorbed in the stomach and/or intestine, but cause irritation to the stomach and/or intestine and therefore are difficult to tolerate. In such a situation, a subject could benefit if the bioavailability of the therapeutic agent were enhanced or if more of the therapeutic agent were absorbed directly into the blood stream; if less therapeutic agent is administered there will clearly be less chance of causing irritation to the stomach and/or intestine. Thus compositions of the invention are envisaged which comprise therein two or more therapeutic agents.

In general, the composition may include from about 0.01% to about 50% by weight of the therapeutic agent e.g., about 0.01, 0.02, 0.05, 1, 2, 3, 4, 5, 6, 7, 8, 9, 10, 15, 20, 25, 30, 35, 40, 45, or 50% by weight. The maximum included in the composition is often in the range of about 6%-33% by weight of the therapeutic agent.

### Medium Chain Fatty Acid Salt:

The compositions described herein include the salt of a medium chain fatty acid or a derivative thereof in a solid form. For example, the salt of the medium chain fatty acid is in the form of a particle such as a solid particle. In some embodiments, the particle may be characterized as a granulated particle. In at least some embodiments, the solid form may generally result from a spray-drying or evaporation process. In preferred embodiments, the salt of the medium chain fatty acid is in the same particle as the therapeutic agent. For example, the therapeutic agent and the salt of the medium chain fatty acid can be prepared together by first preparing a solution such as an aqueous solution comprising both the therapeutic agent and the salt of the medium chain fatty acid and co-lyophilizing the solution to provide a solid form or particle that comprises both the therapeutic agent and the salt of the medium chain fatty acid (and other ingredients). As described above, the resulting solid particles are associated with a hydrophobic medium. For example, the solid particles may be suspended or immersed in a hydrophobic medium

In different embodiments of the compositions described herein the medium chain fatty acid salt and the matrix forming polymer (see below) may be in the same particle or in a different particle than that of the API. It was found that bioavailability of a cargo compound was lower if the medium chain fatty acid was in a different particle than the therapeutic agent i.e. there was improved bioavailability if the medium chain fatty acid salt and the cargo compound were dried after solubilization together in the hydrophilic fraction. In one embodiment the medium chain fatty acid salt, the matrix forming polymer and the cargo compound are all in the same particle in the final powder.

Medium chain fatty acid salts include those having a carbon chain length of from about 6 to about 14 carbon atoms. Examples of fatty acid salts are sodium hexanoate, sodium heptanoate, sodium octanoate (also termed sodium caprylate), sodium nonanoate, sodium decanoate, sodium undecanoate, sodium dodecanoate, sodium tridecanoate, and sodium tetradecanoate. In some embodiments, the medium chain fatty acid salt contains a cation selected from the group consisting of potassium, lithium, ammonium and other monovalent cations e.g., the medium chain fatty acid salt is selected from lithium octanoate or potassium octanoate or arginine octanoate or other monovalent salts of the medium chain fatty acids. The inventors found that raising the amount of medium chain fatty acid salt increased the bioavailability of the resulting formulation. In particular, raising the amount of medium chain fatty acid salt, in particular sodium octanoate, above 10% to a range of about 12% to 15% increased the bioavailability of the therapeutic agents in the pharmaceutical compositions described herein.

In general, the amount of medium chain fatty acid salt in the compositions described herein may be from 10% up to about 50% by weight of the bulk pharmaceutical composition. For example, the medium chain fatty acid salt may be present at an amount of about 10% -50%, or at an amount of about 10%-20% or about 10-15% or about 15-20%, preferably about 11%-40% most preferably about 11%-28% by weight for example at about 12%-13%, 13%-14%, 14%-15% , 15%-16%, 16%-17%, 17%-18%, 18%-19%, 19%-20%-21%, 21%-22%, 22%-23%, 23%-24%,2 4%-25%, 25%-26%, 26%-27%, or 27%-28% by weight of the bulk pharmaceutical composition. In other embodiments the medium chain fatty acid salt may be present at an amount of at least about 11%, at least aboutl2%, at least about 13%, at least aboutl4%, at least about 15% at least about 16%,at least about 17%, at least about 18%, at least about 19%, at least about 20%, at least about 21%, at least about 22%, at least about 23%, at least about 24%, at least about 25%, at least about 26%, at least about 27% or at least about 28% by weight of the bulk pharmaceutical composition. In specific embodiments the medium chain fatty acid salt (sodium, potassium, lithium or ammonium salt or a mixture thereof) is present at about 12% -21% by weight of the bulk pharmaceutical composition preferably 11%-18% or about 11%-17% or 12%-16% or 12%-15% or 13%-16% or 13%-15% or 14%-16% or 14%-15% or 15%-16% or most preferably 15% or 16%. In specific embodiments the medium chain fatty acid salt (having a carbon chain length of from about 6 to about 14 carbon atoms particularly 8, 9 or 10 carbon atoms) is present at about 12% -21% by weight of the bulk pharmaceutical composition preferably 11%-18% about 11%-17% or 12%-16% or 12%-15% or 13%-16% or 13%-15% or 14%-16% or 14%-15% or 15%-16% or most preferably 15% or 16%. In specific embodiments the medium chain fatty acid salt (for example salts of octanoic acid, salts of suberic acid, salts of geranic acid) is present at about 12% -21% by weight of the bulk pharmaceutical composition preferably 11%-18% about 11%-17% or 12%-16% or 12%-15% or 13%-16% or 13%-15% or 14%-16% or 14%-15% or 15%-16% or most preferably 15% or 16%. In certain embodiments the medium chain fatty acid salt is present in the solid powder at an amount of 50% to 90%, preferably at an amount of 70% to 80%.

One embodiment of the invention comprises a composition comprising a suspension which consists essentially of an admixture of a hydrophobic medium and a solid form wherein the solid form comprises a therapeutically effective amount of a therapeutic agent, at least one salt of a medium chain fatty acid and a matrix forming polymer, and wherein the medium chain fatty acid salt is not a sodium salt. The salt may be the salt of another cation e.g., lithium, potassium or ammonium; an ammonium salt is preferred.

### Matrix Forming Polymer:

In certain embodiments the composition of the invention comprises a suspension which comprises an admixture of a hydrophobic medium and a solid form wherein the solid form comprises a therapeutically effective amount of a therapeutic agent, at least one salt of a medium chain fatty acid and a matrix forming polymer. In certain embodiments the composition comprises a suspension which consists essentially of an admixture of a hydrophobic medium and a solid form wherein the solid form comprises a therapeutically effective amount of a therapeutic agent(e.g., CCK-8), at least one salt of a medium chain fatty acid and a matrix forming polymer. The matrix forming polymer is preferably present in the composition at an amount of about 0.5% to about 10% by weight, most preferably at an amount of about 1% to about 10% by weight.

Matrix forming polymers include polyvinylpyrrolidone (PVP) and cross-linked PVP (cross-povidones); ionic polysaccharides (for example hyaluronic acid/hyaluronates and alginic acid/alginates); neutral polysaccharides (for example dextran, methyl cellulose and hydroxypropyl methylcellulose (HPMC)); linear polyacrylic acid polymers including polymethacrylic acid polymers; cross-linked polyacrylic acid polymers (carbomers); amino-polysaccharides (e.g., chitosans); S-containing polymers (thiomers); and high molecular weight linear and bridged organic alcohols (for example linear polyvinyl alcohol).

Carbomer is a generic name for cross-linked polymers of acrylic acid; carbomers may be homopolymers of acrylic acid, cross-linked with, for example, an allyl ether pentaerythritol, or allyl ether of sucrose or allyl ether of propylene or allyl sucrose or other sugars or allyl pentaerythritol or a polyalkenyl ether or divinyl glycol.

In particular embodiments the matrix forming polymer is polyvinylpyrrolidone (PVP), carbomer, polyvinyl alcohol (PVA), dextran, alginate salt, hyaluronate salt or polyacrylic acid salt or a combination thereof. In certain particular embodiments the matrix forming polymer is polyvinylpyrrolidone (PVP), Carbopol polymer or polyvinyl alcohol (PVA) or a combination thereof.

In particular embodiments the polyvinylpyrrolidone is present in the composition at an amount of about 2% to about 20% by weight, preferably at an amount of about 3% to about 18% by weight, more preferably at an amount of about 5% to about 15% by weight, most preferably at an amount of about 10% by weight. In certain particular embodiments the polyvinylpyrrolidone is PVP-12 and/or has a molecular weight of about 3000, and is present in the composition at an amount of about 2% to about 20% by weight, preferably at an amount of about 5% to about 15% by weight, most preferably at an amount of about 10% by weight.

In one aspect of the invention the matrix forming polymer is a cross-linked acrylic acid polymer (also termed carbomer). Carbopol polymers are examples of cross-linked polymers of acrylic acid. The viscosity of the cross-linked acrylic acid polymer is about 2000-80000 cP, preferably 4000-65000, most preferably 25000-45000 cP; the viscosity is measured in cP, 0.5% solution at pH7.5. In one particular aspect of the invention the cross-linked acrylic acid polymer is an allyl sucrose-linked carbomer, of viscosity about 29000 to about 40000, particularly Carbopol 934P. The cross-linked acrylic acid polymers may be present in the composition at an amount of about 0.1% to about 6% by weight, preferably at an amount of about 0.5% to about 4% by weight, e.g., at an amount of about 1% or about 2% or about 3% by weight.

In another aspect of the invention, the matrix forming polymer is polyvinyl alcohol of molecular weight 10000-60000 Da, preferably 20000-30000 Da. In particular embodiments the polyvinyl alcohol is polyvinyl alcohol of molecular weight of about 27000 Da, and may be present in the composition at an amount of about 0.1% to about 6% by weight, preferably at an amount of about 0.5% to about 4% by weight, e.g., at an amount of about at an amount of about 1%, about 2%, or about 3% by weight.

Glucose and/or other sugars and/or mannitol may be substituted in certain embodiments instead of a matrix forming polymer.

### Protease Inhibitors:

It is generally accepted in the art of delivery of proteins, polypeptides and peptides that protease inhibitors normally have to be added to the formulation to prevent degradation of the API. However in formulations of the instant invention it is not normally necessary to add protease inhibitors. The formulations of the invention appear to confer stability of the therapeutic agent to protease degradation within the time-frame of activity i.e. the formulations of the invention are apparently environment inhibitory for enzyme activity. Additionally, the inventors performed an experiment wherein the protease inhibitor aprotinin was added to a formulation and this had no beneficial effect on activity. A similar experiment was performed where the protease inhibitor ε-aminocaproic acid was added to a formulation and this too had no beneficial effect on activity. In these formulations the polypeptide was growth hormone. Therefore, in some embodiments, a pharmaceutical composition described herein is substantially free of a protease inhibitor. In other embodiments, a protease inhibitor is present, in particular an endopeptidase inhibitor.

### Hydrophilic Fraction:

In embodiments of the invention, the above compounds, including the therapeutic agent, the medium chain fatty acid salt and the matrix forming polymer (or substitute) are solubilized in an aqueous medium and then dried to produce a powder. The drying process may be achieved for example by lyophilization or granulation or by spray-drying or by other means. The powder obtained is termed the “hydrophilic fraction”. In the hydrophilic fraction water is normally present at an amount of less than 6% after drying, and the water in the final bulk composition comprises residual water from the hydrophilic fraction.

Lyophilization may be carried out as shown in the Examples herein and by methods known in the art e.g., as described in Lyophilization: Introduction and Basic Principles, Thomas Jennings, published by Interpharm/CRC Press Ltd (1999, 2002) The lyophilizate may optionally be milled (e.g., below 150 micron) or ground in a mortar. During industrial production the lyophilizate is preferably milled before mixing of the hydrophilic fraction and the hydrophobic(lipophilic) medium in order to produce batch-to-batch reproducibility.

Granulation may be carried out as shown in the Examples herein and by methods known in the art e.g., as described in Granulation, Salman et al., eds, Elsevier (2006) and in Handbook of Pharmaceutical Granulation Technology, 2^{nd} edition, Dilip M. Parikh, ed., (2005). Various binders may be used in the granulation process as described in the previous two references.

Spray-drying may be carried out by methods known in the art e.g., as described by Patel et al. (2009) Indian Journal of Science and Technology 2(10) 44-47 and by Shabde, Vikram (2006) Ph.D. thesis, Texas Tech University.

### Hydrophobic (Lipophilic) Medium:

Oil: As described above, in the compositions of the invention described herein the therapeutic agent and the medium chain fatty acid salt are in intimate contact or association with a hydrophobic (oily) medium. For example, one or both may be coated, suspended, immersed or otherwise in association with a hydrophobic(lipophilic) medium. Suitable hydrophobic mediums can contain, for example, aliphatic, cyclic or aromatic molecules. Examples of a suitable aliphatic hydrophobic medium include, but are not limited to, mineral oil, fatty acid monoglycerides, diglycerides, triglycerides, ethers, esters, and combinations thereof. Examples of a suitable fatty acid are octanoic acid, decanoic acid and dodecanoic acid, also C7 and C9 fatty acids and di-acidic acids such as sebacic acid and suberic acid, and derivatives thereof. Examples of triglycerides include, but are not limited to, long chain triglycerides, medium chain triglycerides, and short chain triglycerides. For example, the long chain triglyceride can be castor oil or olive oil, and the short chain triglyceride can be glyceryl tributyrate and the medium chain triglyceride can be glyceryl tricaprylate or coconut oil. Monoglycerides are considered to be surfactants and are described below. Exemplary esters include ethyl isovalerate and butyl acetate. Examples of a suitable cyclic hydrophobic medium include, but are not limited to, terpenoids, cholesterol, cholesterol derivatives (e.g., cholesterol sulfate), and cholesterol esters of fatty acids. A non-limiting example of an aromatic hydrophobic medium includes benzyl benzoate.

In some embodiments of the compositions described herein, it is desirable that the hydrophobic medium include a plurality of hydrophobic molecules. In some embodiments of the compositions described herein the hydrophobic medium also includes one or more surfactants (see below).

Surface Active Agents (surfactants): The compositions of this invention described herein can further include a surface active agent. For example, the surface active agent can be a component of the hydrophobic medium as described above, and/or the surface active agent can be a component of a solid form as described above, for example in the solid form or particle that includes the therapeutic agent.

Suitable surface active agents include ionic and non-ionic surfactants. Examples of ionic surfactants are lecithin (phosphatidyl choline), bile salts and detergents. Examples of bile salts are sodium taurocholate, sodium deoxycholate, sodium glycocholate, sodium chenodeoxycolate, sodium cholate, sodium lithocholate, in particular sodium taurocholate. Examples of non-ionic surfactants include monoglycerides, cremophore, a polyethylene glycol fatty alcohol ether, a sorbitan fatty acid ester, a polyoxyethylene sorbitan fatty acid ester, Solutol HS15, a poloxamer , alkyl-saccharides (e.g., octyl glycoside, tetra decyl maltoside), and a combination thereof. Examples of monoglycerides are glyceryl monocaprylate (also termed glyceryl monooctanoate), glyceryl monodecanoate, glyceryl monolaurate, glyceryl monomyristate, glyceryl monostearate, glyceryl monopalmitate, and glyceryl monooleate.

Examples of sorbitan fatty acid esters include sorbitan monolaurate, sorbitan monooleate, and sorbitan monopalmitate (Span 40), or a combination thereof. Particular examples of polyoxyethylene sorbitan fatty acid esters include polyoxyethylene sorbitan monooleate (Tween 80), polyoxyethylene sorbitan monostearate, polyoxyethylene sorbitan monopalmitate or a combination thereof. The commercial preparations of monoglycerides that were used also contain various amounts of diglycerides and triglycerides. Compositions described herein including a surface active agent generally include less than about 12% by weight of total surface active agent (e.g., less than about 10%, less than about 8%, less than about 6%, less than about 4%, less than about 2%, or less than about 1%). In particular embodiments of the invention the total sum of all the surfactants is about 6-7% by weight in the composition. In certain embodiments the surfactants include Tween 80 at about 2% by weight and glyceryl monocaprylate at about 4-5% by weight. In certain embodiments the surfactants include lecithin at about 6% by weight in the hydrophobic(lipophilic) medium. In particular embodiments the surfactants include lecithin in the hydrophobic (lipophilic) medium and a bile salt in particular sodium taurocholate in the hydrophilic (solid) faction.

### Methods of Making Pharmaceutical Compositions and the Compositions Produced:

Also included in the invention are methods of producing the compositions described herein. Thus one embodiment of the invention is a process for producing a pharmaceutical composition which comprises preparing a water-soluble composition comprising a therapeutically effective amount of at least one therapeutic agent (as described herein), a medium chain fatty acid salt and a matrix forming polymer or substitute (as described herein), and optionally a surfactant and optionally a stabilizer, drying the water soluble composition to obtain a solid powder, and suspending the solid powder in a hydrophobic medium, to produce a suspension containing in solid form the therapeutic agent, the medium chain fatty acid salt and the matrix forming polymer, and optionally a surfactant and optionally a stabilizer, thereby producing the pharmaceutical composition; in certain aspects of the invention the pharmaceutical composition contains about 10% -15% by weight of medium chain fatty acid salt; see FIG. 1.

One embodiment is a process for producing a pharmaceutical composition which comprises providing a solid powder of a therapeutically effective amount of at least one therapeutic agent, a solid powder comprising a medium chain fatty acid salt and a solid powder comprising matrix forming polymer, and suspending the solid powders in a hydrophobic medium, to produce a suspension containing in solid form the therapeutic agent and the medium chain fatty acid salt, thereby producing the pharmaceutical composition, wherein the pharmaceutical composition contains 10% or more by weight of medium chain fatty acid salt. In certain aspects of the invention a surfactant as described herein is present; it is present in the hydrophobic medium and/or in the solid form. In certain aspects of the invention a stabilizer as described herein is present; it is present in the hydrophobic medium and/or in the solid form, in particular in the solid form.

In a particular embodiment of the processes and compositions described herein the matrix forming polymer is selected from the group comprising cross-linked acrylic acid polymer, polyvinyl alcohol polymer of molecular weight 10000-70000 Da and cross-linked PVP (cross-povidones) and hyaluronic acid and salts thereof. In certain embodiments of the processes and compositions described herein the matrix forming polymer is cross-linked acrylic acid polymer or polyvinyl alcohol polymer of molecular weight 10000-70000 Da.

In one embodiment of the processes and compositions described herein, the water-soluble composition is an aqueous solution. In certain embodiments the drying of the water-soluble composition is achieved by lyophilization or by granulation or by spray-drying or by other means. In the granulation process a binder may be added to the water soluble composition before drying. In certain embodiments the drying step removes sufficient water so that the water content in the pharmaceutical composition is lower than about 6% by weight, about 5% by weight, about 4% by weight, about 3% or about 2% or about 1% by weight. In certain embodiments of the processes and compositions described herein the drying step removes an amount of water so that the water content in the solid powder is lower than 6% or 5% or 4% or 3% or preferably lower than 2% by weight. The water content is normally low and the water may be adsorbed to the solid phase during lyophilization i.e. the water may be retained by intermolecular bonds. In certain embodiments the water soluble composition additionally comprises a stabilizer. In preferred embodiments of the processes and compositions described herein the hydrophobic medium is castor oil or glyceryl tricaprylate or glyceryl tributyrate or a combination thereof and may additionally contain octanoic acid; in certain embodiments the hydrophobic medium comprises an aliphatic, olefinic, cyclic or aromatic compound, a mineral oil, a paraffin, a fatty acid such as octanoic acid, a monoglyceride, a diglyceride, a triglyceride, an ether or an ester, or a combination thereof. In certain embodiments of the processes and compositions described herein the triglyceride is a long chain triglyceride, a medium chain triglyceride preferably glyceryl tricaprylate or coconut oil or a short chain triglyceride preferably glyceryl tributyrate, and the long chain triglyceride is castor oil, or a combination thereof. In certain embodiments of the processes and compositions described herein the hydrophobic medium comprises castor oil or glyceryl tricaprylate or glyceryl tributyrate or a combination or mixture thereof, and may additionally comprise octanoic acid. In certain embodiments of the processes and compositions described herein the hydrophobic medium comprises glyceryl tricaprylate or a low molecular weight ester for example ethyl isovalerate or butyl acetate. In certain embodiments of the processes and compositions described herein the main component by weight of the hydrophobic medium is castor oil and may additionally comprise glyceryl tricaprylate. In certain embodiments of the processes and compositions described herein the main component by weight of the hydrophobic medium is glyceryl tricaprylate.

A basic formulation is provided as an embodiment wherein the hydrophobic medium consists essentially of castor oil, glyceryl monooleate and glyceryl tributyrate; in a further embodiment of the basic formulation the hydrophilic fraction consists essentially of therapeutic agent, PVP-12 and sodium octanoate.

A particular formulation is provided as an embodiment wherein the hydrophobic medium consists essentially of glyceryl tricaprylate, castor oil, glyceryl monocaprylate, and Tween 80, and the hydrophilic fraction consists essentially of therapeutic agent (e.g., octreotide), PVP-12 and sodium octanoate. Another particular formulation is provided as an embodiment wherein the hydrophobic medium comprises glyceryl tricaprylate, castor oil, glyceryl monocaprylate, and Tween 80, and the hydrophilic fraction comprises therapeutic agent (e.g., octreotide), PVP-12 and sodium octanoate. In certain embodiments the hydrophobic medium consists essentially of glyceryl tricaprylate and in certain embodiments additionally contains castor oil and/or glyceryl monocaprylate.

A particular formulation is provided as an embodiment wherein the solid form comprises a therapeutically effective amount of a therapeutic agent, sodium octanoate, PVP-12 and at least one surfactant, preferably a bile salt, preferably sodium taurocholate, and a stabilizer; and wherein the hydrophobic medium comprises glyceryl tricaprylate, and at least one surfactant, preferably lecithin.

In certain particular aspects of the processes and compositions of the invention, PVP-12 is replaced by another matrix forming polymer such as Carbopol 934P or PVA. In certain aspects of the processes and compositions of the invention, the matrix forming polymer is selected from the group consisting of polyvinylpyrrolidone, carbomer (e.g., Carbopol polymer), polyvinyl alcohol, dextran, alginate salt, hyaluronate salt, and polyacrylic acid salt or a combination thereof. In certain particular aspects of the processes and compositions of the invention, the matrix forming polymer is selected from the group consisting of polyvinylpyrrolidone, Carbopol polymer and polyvinyl alcohol or a combination thereof. In certain processes and compositions of the invention the polyvinylpyrrolidone is PVP-12, preferably having a molecular weight of about 3000, and is present in the composition at an amount of about 2% to about 20% by weight, preferably at an amount of about 5% to about 15% by weight, most preferably at an amount of about 10% by weight. In certain processes and compositions of the invention the Carbopol polymer is preferably Carbopol 934P, and is present in the composition at an amount of about 0.1% to about 20% by weight, preferably at an amount of about 0.5% to about 10% by weight, for example at an amount of about 1% or 2% or 3% by weight. In certain processes and compositions of the invention the polyvinyl alcohol is preferably polyvinyl alcohol of molecular weight of about 27000 Da, and is present in the composition at an amount of about 0.1% to about 20% by weight, preferably at an amount of about 0.5% to about 10% by weight, for example at an amount of about 1% or 2% or 3% by weight

In certain embodiments the composition comprises a suspension which consists essentially of an admixture of a hydrophobic medium and a solid form wherein the solid form comprises a therapeutically effective amount of a therapeutic agent, a matrix forming polymer and at least one salt of a medium chain fatty acid, and wherein the medium chain fatty acid salt is present in the composition at an amount of 10% or more by weight. In certain embodiments the hydrophobic medium consists essentially of castor oil, glyceryl monooleate and glyceryl tributyrate; or the hydrophobic medium consists essentially of glyceryl tricaprylate and glyceryl monocaprylate; or the hydrophobic medium consists essentially of castor oil, glyceryl tricaprylate and glyceryl monocaprylate. In certain embodiments the hydrophobic medium comprises a triglyceride and a monoglyceride and in certain particular embodiments the monoglyceride has the same fatty acid radical as the triglyceride. In certain of these embodiments the triglyceride is glyceryl tricaprylate and the monoglyceride is glyceryl monocaprylate. In certain embodiments the medium chain fatty acid salt in the water-soluble composition has the same fatty acid radical as the medium chain monoglyceride or as the medium chain triglyceride or a combination thereof. In certain of these embodiments the medium chain fatty acid salt is sodium caprylate (sodium octanoate) and the monoglyceride is glyceryl monocaprylate and the triglyceride is glyceryl tricaprylate.

Many of the compositions described herein comprise a suspension which comprises an admixture of a hydrophobic medium and a solid form wherein the solid form comprises a therapeutically effective amount of a therapeutic agent, a matrix forming polymer and at least one salt of a medium chain fatty acid, and wherein the medium chain fatty acid salt is present in the composition at an amount of 10% or more by weight.

In all the formulations described herein, the percentages recited are weight/weight. The solid form in the formulations described herein may be a particle (e.g., consist essentially of particles, or consists of particles). The particle may be produced by lyophilization or by granulation or by spray-drying or by other means.

In a particular embodiment the formulation consists essentially of a suspension which comprises an admixture of a hydrophobic medium and a solid form wherein the solid form comprises a therapeutically effective amount of a therapeutic agent and about 10-20% preferably 15% medium chain fatty acid salt preferably sodium octanoate, and about 2-10% preferably 10% PVP-12, and optionally a surfactant preferably a bile salt preferably sodium taurocholate and optionally a stabilizer; and wherein the hydrophobic medium comprises about 20-80% , preferably 30-70% triglyceride preferably glyceryl tricaprylate or glyceryl tributyrate or castor oil or a mixture thereof, about 3-10% surfactants, preferably about 6%, preferably lecithin or glyceryl monocaprylate or Tween 80 or a combination thereof, and about 1% water; in particular embodiments the therapeutic agent is present at an amount of less than 33%, or less than 25%, or less than 10%, or less than 1% or less than 0.1%.

In a further embodiment the formulation consists essentially of a suspension which comprises an admixture of a hydrophobic medium and a solid form wherein the solid form comprises a therapeutically effective amount of a therapeutic agent and about 10-20% preferably 15% medium chain fatty acid salt preferably sodium octanoate and about 0.5-10% preferably 1-2% Carbopol 934P or PVA (27000 Da) ; and wherein the hydrophobic medium comprises about 20-80% , preferably 30-70% medium or short chain triglyceride preferably glyceryl tricaprylate or glyceryl tributyrate, about 0-50% preferably 0-30% castor oil, about 3-10% surfactants, preferably about 6%, preferably glyceryl monocaprylate and Tween 80, and about 1% water; in particular embodiments the therapeutic agent is present at an amount of less than 33%, or less than 25%, or less than 10%, or less than 1% or less than 0.1%.

In a particular embodiment the formulation consists essentially of a suspension which comprises an admixture of a hydrophobic medium and a solid form wherein the solid form comprises a therapeutically effective amount of a therapeutic agent and about 10-20% preferably 15% medium chain fatty acid salt preferably sodium octanoate, and about 2-10% preferably 10% PVP-12; and wherein the hydrophobic medium comprises about 20-80%, preferably 30-70% medium or short chain triglyceride preferably glyceryl tricaprylate or glyceryl tributyrate, preferably about 30-80% glyceryl tricaprylate; in particular embodiments the therapeutic agent is present at an amount of less than 33%, or less than 25%, or less than 10%, or less than 1% or less than 0.1%.

In a particular embodiment the formulation consists essentially of a suspension which comprises an admixture of a hydrophobic medium and a solid form wherein the solid form comprises a therapeutically effective amount of a therapeutic agent and about 10-20% preferably 15% medium chain fatty acid salt preferably sodium octanoate, about 2-10% preferably 10% PVP-12, optionally about 0.1-2% preferably 0.5% surfactant, preferably bile salt, preferably sodium taurocholate, and optionally a stabilizer; and wherein the hydrophobic medium comprises about 20-80% , preferably 30-70% medium or short chain triglyceride preferably glyceryl tricaprylate or glyceryl tributyrate, preferably about 30-80% glyceryl tricaprylate and about 3-10% surfactants, preferably about 6%, preferably lecithin; in particular embodiments the therapeutic agent is present at an amount of less than 33%, or less than 25%, or less than 10%, or less than 1% or less than 0.1%.

In embodiments wherein the therapeutic agent is CCK-8, including the above embodiments, it is preferably present at an amount between 0.03 to 3.3%, more preferably at an amount between 0.03 to 0.2%.

In some embodiments, the therapeutic agent within the formulations of the invention is stable over an extended period of time. The chemical and physical state of the formulation is stable. Once administered to the intestine the therapeutic agent is protected from damage by the GI environment since the formulations are oil-based. Thus, a separate local environment is created in the intestine where the therapeutic agent is contained in oil droplets, which confers stability in vivo.

In certain embodiments the process produces a composition which consists essentially of a therapeutic agent, a medium chain fatty acid salt, a matrix forming polymer and a hydrophobic medium. In embodiments of the invention the solid powder (solid form) consists essentially of a therapeutic agent, a medium chain fatty acid salt and a matrix forming polymer. Further embodiments of the invention are pharmaceutical compositions produced by the process describe herein. In certain pharmaceutical compositions the therapeutic agent is a protein, a polypeptide, a peptide, a glycosaminoglycan, a polysaccharide, a small molecule or a polynucleotide and in particular embodiments the therapeutic agent is insulin, growth hormone, parathyroid hormone or analogs thereof such as teriparatide, interferon-alfa (IFN-α), a low molecular weight heparin, leuprolide, fondaparinux, octreotide, exenatide, terlipres sin, vancomycin, gentamicin, cholecystokinin or analogs thereof such as cholecystokinin-8 (CCK-8) and analogs thereof, calcitonin and aliskiren, and salts thereof. Particular embodiments of the invention comprise an oral dosage form comprising the pharmaceutical composition, in particular an oral dosage form which is enteric coated.

Further embodiments of the invention comprise a capsule or tablet containing the compositions of the invention, and in various embodiments the capsule is a hard gel or a soft gel capsule, and generally the capsule or tablet is enteric-coated.

Other embodiments of the invention comprise a rectal dosage form comprising the pharmaceutical composition, in particular a suppository, or a buccal dosage form. A kit comprising instructions and the dosage form is also envisaged.

The therapeutic agent or medium chain fatty acid salt or matrix forming polymer, or any combination of therapeutic agent and other components, can be prepared in a solution of a mixture (e.g., forming an aqueous solution or mixture) which can be lyophilized together and then suspended in a hydrophobic medium. Other components of the composition such as a stabilizer or a surfactant, can also be optionally lyophilized or added during reconstitution of the solid materials.

In some embodiments, the therapeutic agent is solubilized in a mixture, for example, including one or more additional components such as a medium chain fatty acid salt, a matrix forming polymer, a stabilizer and/or a surface active agent, and the solvent is removed to provide a resulting solid powder (solid form), which is suspended in a hydrophobic medium. In some embodiments, the therapeutic agent and/or the medium chain fatty acid salt and/or the matrix forming polymer may be formed into a granulated particle that is then associated with the hydrophobic medium (for example suspended in the hydrophobic medium or coated with the hydrophobic medium).

The compositions described herein are substantially free of any “membrane fluidizing agents”. For example the compositions preferably include no membrane fluidizing agents but certain embodiments may include for example less than 1% or less than 0.5% or less than 0.1% by weight of membrane fluidizing agents. “Membrane fluidizing agents” are defined as medium chain alcohols which have a carbon chain length of from 4 to 15 carbon atoms (e.g., including 5 to 15, 5 to 12, 6, 7, 8, 9, 10, or 11 carbon atoms). For example, a membrane fluidizing agent can be a linear (e.g., saturated or unsaturated), branched (e.g., saturated or unsaturated), cyclical (e.g., saturated or unsaturated), or aromatic alcohol. Examples of suitable linear alcohols include, but are not limited to, butanol, pentanol, hexanol, heptanol, octanol, nonanol, decanol, undecanol, dodecanol, tridecanol, tetradecanol, and pentadecanol. Examples of branched alcohols include, but are not limited to, geraniol, farnesol, rhodinol, citronellol. An example of a cyclical alcohol includes, but is not limited to, menthol, terpineol, myrtenol, perillyl and alcohol. Examples of suitable aromatic alcohols include, but are not limited to, benzyl alcohol, 4-hydroxycinnamic acid, thymol, styrene glycol, and phenolic compounds. Examples of phenolic compounds include, but are not limited to, phenol, m-cresol, and m-chlorocresol. If desired, the pharmaceutical composition may also contain minor amounts of non-toxic auxiliary substances such pH buffering agents, and other substances such as for example, sodium acetate and triethanolamine oleate. In at least one embodiment, a therapeutic agent, such as a protein, may be chemically modified to enhance its half-life in circulation. For example, the therapeutic agent may undergo a process such as pegylation.

In some embodiments the process for producing a pharmaceutical composition comprises preparing a water-soluble composition comprising a therapeutically effective amount of at least one therapeutic agent, a medium chain fatty acid salt and a matrix forming polymer, drying the water soluble composition to obtain a solid powder, and dissolving the solid powder in a solution consisting essentially of octanoic acid, thereby producing the pharmaceutical composition. At low concentration of solids the resulting formulation is a solution. At higher concentration of solids the saturation threshold is reached after which a suspension is obtained e.g., the aliskiren solution exemplified. In some embodiments, the solid form may be a particle (e.g., consist essentially of particles, or consists of particles). In some embodiments, the particle may be produced by lyophilization or by granulation or by spray-drying or by other means. In some embodiments of this process the octanoic acid is present in the composition at a level of about 50% to about 90% or at a level of about 65 to about 85%, preferably about 70%. In some embodiments of this process the fatty acid salt is sodium octanoate; in further embodiments of this process the medium chain fatty acid salt is present in the composition at an amount of about 11% to about 40% by weight or at an amount of about 11% to about 28% by weight or at an amount of about 15% by weight. The matrix forming polymer may be present as described above. The composition may in addition include one or more surfactants and optionally a stabilizer as described above. The pharmaceutical products of these processes are further embodiments of the invention e.g., a composition containing octanoic acid at a level of about 60% to about 90% or at a level of about 60 to about 85% and a fatty acid salt, preferably sodium octanoate, present in the composition at an amount of about 11% to about 40% by weight or at an amount of about 11% to about 28% by weight or at an amount of about 15% by weight; matrix forming polymer present in the composition at an amount of about 2% to about 20% by weight or preferably an amount of about 5% to about 15% by weight, preferably at an amount of about 10% by weight. In one aspect of the invention the matrix forming polymer may be PVA or Carbopol polymer or other matrix forming polymers at an amount of 0.5-10% preferably 1-2%, and surfactants as described above. There also may be small quantities of other hydrophobic constituents as described above.

Capsules and tablets: Preferred pharmaceutical compositions are oral dosage forms or suppositories. Exemplary dosage forms containing the bulk drug product include gelatin (hard gel or soft gel) or vegetarian capsules like starch hydroxylpropyl-methylcellulose (“HPMC”) capsules; the capsules may be enteric coated. An enteric coating is resistant to stomach acid thus allowing intact capsule or tablet to pass the stomach and reach the intestine in which it dissolves in the less acidic area of the intestines, thus releasing the therapeutic agent. Examples of enteric coatings are Acryl-EZE™ (a methacrylic acid copolymer type C), Opadry™ Enteric series 91 (a polyvinyl acetate phthalate) Sureteric™ (also a polyvinyl acetate phthalate), Opadry™ Enteric series 94 (methacrylic acid-methyl methacrylate 1:1 copolymer), Opadry™ Enteric series 95 (methacrylic acid-methyl methacrylate 1:2 copolymer)—all from Colorcon; Eudragit™ series (polymethylacrylates) from Evonik Rohm Gmbh; Aquacoat CPD (cellulose acetate phthalate) from FMC Biopolymer, USA; Eastman C-A-P Cellulose Ester (cellulose acetate phthalate) from Eastman; HPMCP—50(hydroxypropyl methylcellulose phthalate) and HPMCAS Shin-Etsu AQOAT (hydroxypropyl methylcellulose acetate succinate)—both from Shin Etsu, Japan; and CMEC (carboxymethyl cellulose) from Freund, Japan.

Capsules which may be used to encapsulate the compositions of this invention are known in the art and are described for example in Pharmaceutical Capsules edited by Podczech and Jones, Pharmaceutical Press (2004) and in Hard gelatin capsules today—and tomorrow, 2nd edition, Steggeman ed published by Capsugel Library (2002). Capsules can be coated with the same materials as tablets (sometimes a sub-coat or binder for better adhesion of enteric polymer is needed). Tablets comprising solid forms of the bulk drug product, and tabletted with suitable excipients as known in the art, are also envisaged; the tablets may be enteric coated. An oral dosage form according to the invention comprises additives or excipients that are suitable for the preparation of the oral dosage form according to the present invention and may be prepared as described herein. A kit comprising instructions and the dosage form is also envisaged.

Additional formulations: The compositions of the invention may be formulated using additional methods known in the art, for example as described in the following publications: Pharmaceutical Dosage Forms Vols 1-3 ed. Lieberman, Lachman and Schwartz, published by Marcel Dekker Inc, New York (1989); Water-insoluble Drug Formulation 2^{nd} edition, Liu, editor, published by CRC Press, Taylor and Francis Group (2008); Therapeutic Peptides and Proteins: Formulation, Processing and Delivery Systems, 2^{nd} edition by Ajay K. Banga (author) published by CRC Press, Taylor and Francis Group (2006); Protein Formulation and Delivery, 2^{nd} edition, McNally and Hasted eds, published by Informa Healthcare USA Inc (2008); and Advanced Drug Formulation to Optimize Therapeutic Outcomes, Williams et al eds, published by Informa Healthcare USA (2008).

The compositions of the invention may be formulated using microparticulate technology for example as described in Microparticulate Oral Drug Delivery, Gerbre-Selassie ed., published by Marcel Dekker Inc (1994) and in Dey et al, Multiparticulate Drug Delivery Systems for Controlled Release, Tropical Journal of Pharmaceutical Research, September 2008; 7 (3): 1067-1075.

Methods of treatment: The compositions described herein exhibit effective, enteral delivery of an unaltered biologically active substance (i.e. a therapeutic agent) and thus, have many uses.

In particular, insulin to treat and prevent subjects (patients) suffering from Type II diabetes (prophylaxis of diabetes), and to treat patients suffering from dysglycemia, pre-diabetes and metabolic syndrome and other conditions, may be administered in any oral formulation which confers sufficient bioavailability, in particular in formulations in accordance with one or more embodiments of the invention. One embodiment of the invention is a method of treatment or prevention of a subject suffering from the above conditions where the amount of insulin sufficient to treat the condition is a low dose of insulin formulated within the compositions of the invention. Low dose insulin is provided by less than 300 or less than 200 Units per capsule e.g., 40-200 Units per capsule.

Terlipressin (or other vasopressin analogs) to treat subjects (patients) suffering from hepato-renal syndrome (HRS), including HRS I and II, bleeding esophageal varices, portal hypertension and other conditions may be administered in any oral formulation which confers sufficient bioavailability and in particular in formulations in accordance with one or more embodiments of the invention. Such terlipressin formulations may also be used for primary and secondary prophylaxis of variceal bleeding.

Exenatide to improve glycemic control in subjects suffering from Type II diabetes and to treat other conditions such as obesity and for use in weight management may be administered in administered in any oral formulation which confers sufficient bioavailability and in particular in formulations in accordance with one or more embodiments of the invention.

Interferon-alfa for the treatment of subjects suffering from chronic hepatitis C and chronic hepatitis B and to treat other conditions including cancer may be administered in any oral formulation which confers sufficient bioavailability. A low dose of interferon-alfa may be sufficient when orally administered and thus many of the systemic side-effects of interferon-alfa may be avoided. In particular interferon-alfa may be orally administered in the formulations of the invention, preferably at low dose.

Copaxone to treat subjects suffering from multiple sclerosis and to treat other conditions including inflammatory diseases may administered in any oral formulation which confers sufficient bioavailability and in particular in formulations in accordance with one or more embodiments of the invention.

Desmopressin to treat subjects suffering from primary nocturnal enuresis, central diabetes insipidus (DI) or bleeding disorders (Von Willebrand Disease and Hemopilia A) may be administered in any oral formulation which confers sufficient bioavailability and in particular in formulations in accordance with one or more embodiments of the invention. Oral desmopressin preparations known in the art suffer from extremely low oral bioavailability.

Octreotide: Octreotide was first synthesized in 1979, and is an octapeptide that mimics natural somatostatin pharmacologically, though it is a more potent inhibitor of growth hormone, glucagon and insulin than the natural hormone. Octreotide or other analogs of somatostatin may be administered in accordance with one or more embodiments of the invention for use in treating or preventing a disease or disorder in a subject suffering from a disorder such as acromegaly, abnormal GI motility, flushing episodes associated with carcinoid syndrome, portal hypertension, an endocrine tumor (such as carcinoids, VIPoma), gastroparesis, diarrhea, pancreatic leak or a pancreatic pseudo-cyst. The diarrhea may result from radiotherapy or may occur for example in subjects with vasoactive intestinal peptide-secreting tumors (VIPomas). In addition, patients that undergo pancreatic surgery may suffer from secretion of extrinsic pancreas and are vulnerable to developing pancreatic leak or pseudo-cysts which may be treated by octreotide products of the invention. Some preferred embodiments are directed to a method of treating a subject having a disorder such as acromegaly, abnormal GI motility, flushing episodes associated with carcinoid syndrome, portal hypertension, an endocrine tumor (such as carcinoids, VIPoma), gastroparesis, diarrhea, pancreatic leak or a pancreatic pseudo-cyst, which comprises administering to the subject a composition of the invention, wherein the therapeutic agent is octreotide, in an amount sufficient to treat the disorder. Any octreotide oral formulations which confer sufficient bioavailability may be used for primary and secondary prophylaxis of variceal bleeding, which may be caused by portal hypertension; the varices may be gastric or esophageal. In particular octreotide formulations of the invention may be used for primary and secondary prophylaxis of variceal bleeding, which may be caused by portal hypertension; the varices may be gastric or esophageal. Other uses of octreotide formulations of the invention are in treatment of shock of hypovolemic (e.g., hemorrhagic) or vasodilatory (e.g., septic) origin, hepatorenal syndrome (HRS), cardiopulmonary resuscitation and anesthesia-induced hypotension. Any octreotide oral formulations which confer sufficient bioavailability may be used for the previously mentioned indications for octreotide. Other analogs of somatostatin may be used in the methods and compositions in which octreotide is used and for the previously mentioned indications.

Cholecystokinin-8: Cholecystokinin-8 (CCK-8) is a naturally occurring, 8-amino acid cholecystopancreatic-gastrointestinal peptide hormone in the gastrointestinal system that regulates multiple functions including satiety, gallbladder contraction and gastric emptying. Clinical data indicates that CCK-8 may play a role in obesity, binge eating disorder, and bulimia nervosa. For example, CCK-8 administration reduces meal size and intake in obese patients and CCK-8 levels are disregulated in patients with bulimia nervosa.

CCK-8 is available as Sincalide for parenteral administration under the trade name Kinevac. Sincalide (CAS#25126-32-3), is a synthetically prepared C-terminal octapeptide of cholecystokinin (CCK), with the following amino acid sequence: Asp-Tyr(SO₃H)-Met-Gly-Trp-Met-Asp-Phe-NH₂. Thus CCK-8 is also an analog of CCK. Intravenous CCK-8 has been approved by the FDA since 1976 as Kinevac (sincalide) in an intravenous formulation solely as a diagnostic agent for gallbladder and pancreatic disorders. Octapeptide analogs of CCK-8 which have been chemically modified to produce a more stable or otherwise improved peptide (e.g., a peptidomimetic) are included in the term “CCK-8 analog”, so long as they have the same or similar biological activity as CCK-8, or so long as they bind similar receptors. (See Wank et al, Annals NY Academy Sci., 713:49-66.) Such CCK-8 analogs include AR-R 14294 (formerly FPL 14294; Astra Arcus, formerly Fisons Pharmaceuticals—see Simmons et al. Pharmacol Biochem Behay. 1994 Mar; 47(3):701-8); AR-R 15849 (Astra Arcus; see Simmons et al Pharmacol Biochem Behay. 1998 February; 59(2):439-44.); and U-67827E, which is acetylated and has two norleucine substitutions for the methionines at positions 3 and 6 in the amino acid chain of the peptide (see Moran et al Am. J. Clin. Nutr (1992) 55, 286S-290S).

CCK is released upon food entrance into the duodenum (in particular protein and fat or their metabolites). Secretion of CCK leads to several physiological actions that ensure digestion and absorption of these nutrients: contraction of the gallbladder, secretions of pancreatic enzymes, decrease of gastric emptying, modulation of gastrointestinal motility and suppression of energy intake (See Little et al., 2005 Obesity Reviews, 6, 297-306.)

CCK regulates these physiological actions via neuronal activation of vagal afferent fibers. Alternatively, CCK may act as a hormone and binds to local receptors on target organs (acini pancreatic cells, gallbladder smooth muscles cells); see Zabielski et al., 2003, J Physiol Pharmacol, 54, Suppl 4:81-94. This highly complex regulation may lead to the simultaneous effect of CCK on several organs. For example, high doses of exogenous CCK administration were found to inhibit gastric emptying while CCK-α antagonists accelerate gastric emptying. (Ramkumar D., et al., 2003, Current Opinion in Gastroenterology, 19: 540-545). Similarly, the CK-antagonist, loxiglumide, was shown to reverse the inhibitory effect of both exogenous CCK-8, and fat induced endogenous CCK release, on the subjective feeling of appetite such as hunger and fullness. A direct effect of CCK-α agonism was shown to reduce pre-meal appetite and meal intake in lean humans. Thus, CCK may simultaneously regulate gastric emptying and satiation.

CCK-8 retains the full activity of CCK and exerts similar effects on food intake and satiety in several species (e.g., humans, rats and pigs). Any oral formulations, which have sufficient bioavailability, of CCK and analogs thereof in particular CCK-8 may be administered orally in one or more of the following indications and diagnostic procedures; an example of formulations to be administered are the oral formulations of the invention: (1) to stimulate gallbladder contraction, as may be assessed by contrast agent cholecystography or ultrasonography, or to obtain by duodenal aspiration a sample of concentrated bile for analysis of cholesterol, bile salts, phospholipids, and crystals; (2) to stimulate pancreatic secretion (especially in conjunction with secretin) prior to obtaining a duodenal aspirate for analysis of enzyme activity, composition, and cytology; (3) to accelerate the transit of a barium meal through the small bowel, thereby decreasing the time and extent of radiation associated with fluoroscopy and x-ray examination of the intestinal tract; (4) to produce a feeling of satiety in overweight or obese patients, in order to limit their food intake, and to produce weight loss; in particular to produce weight loss in morbidly obese patients prior to bariatric surgery and to reduce liver size and/or volume prior to bariatric surgery (see below) and to produce weight loss and reduce risk of complications through bariatric surgery; (5) to treat bulimia nervosa and/or binge eating disorder (see below); and (6) as a gall bladder contractor e.g., in a specific set of patients that are on a low-fat diet.

Bariatric surgery, or weight loss surgery, includes a variety of procedures performed on people who are obese e.g., reducing the size of the stomach with an implanted medical device (gastric banding); removal of a portion of the stomach (sleeve gastrectomy or bilio-pancreatic diversion with duodenal switch); resecting and re-routing the small intestines to a small stomach pouch (gastric bypass surgery); and implanting devices that provide electrical stimulation to induce satiety.

Dietary advice given to patients scheduled for bariatric surgery often does not lead to a significant weight change. The literature describes an opposite effect: many patients close to bariatric surgery rather increase their meal intake in expectancy of a reduction in portion size postoperatively, and as a result about 25% of obese patients are not eligible for bariatric surgery. There is a significant unmet need for new agents that promote weight loss in obese patients scheduled for bariatric surgery. Administration of CCK-8 in a 2 week to three month period, preferably 4-8 weeks, prior to surgery should help the patients lose weight.

A common difficulty of bariatric surgery lies in the fatty enlargement of the left lobe of the liver, which acts to obscure the operating field, interfering with adequate visualization of the upper part of the stomach and the gastroesophageal area. Moreover, the enlarged fibrofatty liver may bleed or even fracture during the traumatic retraction necessary to expose the operating field, thus adding to the difficulty of completing a successful procedure. Acute weight loss of body weight (within 4-12 weeks) can reduce the liver size and amount of visceral adipose tissue significantly. The guidelines of the American Society for Metabolic and Bariatric Surgery suggest that preoperative weight loss should be considered in patients in whom reduction of liver volume can improve the technical aspects of surgery. Liver size is routinely measured with ultrasound as part of the screening tests prior to bariatric surgery.

Enlarged liver may be defined as a size which interferes with or obscures the operating field visualization of the upper part of the stomach and the gastroesophageal area. One example of measurement of an enlarged liver is a liver diameter of 20 cm or more when measuring from the left to the right lobe; in this example an “operable” liver size would be considered as 15-16 cm. Acute weight loss thus significantly decreases liver size, and also could improve the metabolic parameters of liver function. CT and MRI can be used for more accurate measurement of the liver and to give a measurement of liver volume. Thus there is a correlation of weight loss to reduction in liver size/fat. See Fris R J et al., 2004, Obesity Surgery (14) 1165-1170; Vitola et al., 2009 Obesity 17(9) 1744-1748; Larson-Meyer e al., 2008, Obesity 16(6) 1355-1362). See also Aberle J. et al., 2009 Obesity Surgery 19: 1504-1507; Lewis M C et al., 2006 Obesity Surgery 16: 697-701.

Thus administration of CCK-8 in a 2 week to three month period, preferably 4-8 weeks, prior to surgery should help the patients lose weight, and, without being bound by theory, also reduce liver size. The surgery may be bariatric surgery or any other surgery or other medical procedures or disease where the enlarged liver is a problem.

Oral CCK-8 may be used to achieve weight loss alone or in conjunction with other active ingredients. Combinations of CCK-8 in conjunction with other anti-obesity drugs, either in the same capsule/ pill or in the same treatment protocol, are also envisaged. Such anti-obesity drugs include orlistat (Xenica™), sibutramine (Meridia™), phendimetrazine tartrate (Bontril™), methamphetamine (Desoxyn™), phentermine (Ionamin™, Adipex-P™), oxyntomodulin, oxyntomodulin analogs, PYY, PYY analogs, GLP-1 and GLP-1 analogs, GLP-1/GIP analog combinations. CCK-8 may also be given in conjunction with a meal replacement program such as Optifast® or Medifast®. Meal replacement programs generally supply their patients with very low calorie shakes or bars that are formulated to contain all the nutrients needed while containing a minimum of calories.

CCK-8 can also be used in regulating appetite and satiety and in turn controlling weight in individuals who are prone to estrogenic weight gain or are engaged in estrogenic hormone therapy. These may include women taking estrogen-containing birth control compositions, as well as those receiving estrogen replacement therapy. Estrogen hormone therapy includes taking selective estrogen receptor modulators (SERMs), for example raloxifene, clomifene and like compounds. Oral CCK-8 may also be used to achieve weight control.

CCK-8 can be quantitatively measured in serum, plasma and other biological fluids by an ELISA kit (e.g., produced by USCNK Life Science Inc., Wuhan, China). CCK-8 can also be measured by immunoprecipitation based LC-MS/MS, LC/MS/MS (LC=Liquid chromatography; MS=mass spectrometry) and by a measurement of stimulation of amylase release from isolated rat pancreatic acini (Young S A et al., 2009, Anal Chem, 81(21):9120-9128; Liddle R A et al., 1985, J Clin Invest, 75:1144-1152). CCK-8 can be also measured by detection of radioactive CCK-8: measurements of ¹²⁵I-CCK samples in lymph by a gamma counter, measurements of ¹²⁵I-CCK using radioimmuno assay (RIA) and measurements of [³H]-CCK8 using a HPLC with a radiometric detector (Chun-Min L et al., 2009, AJP-Regul Integr Comp Physiol, 296:R43-R50; Santangelo A et al., 1998, Br J Nutr 80:521-527; Sheng-Fang S et al., 2002, Biochem Biophys Res Commu, 292:632-638

In addition, pharmacodynamic animal models for measurement of activity and potency of CCK-8 include guinea-pig gallbladder contraction, induction of pancreatic enzyme secretion, effect on lower esophageal sphincter pressure, effect on intestinal motility and energy/food intake; see for example Zabielski et al., 2003, J Physiol Pharmacol 54:293-317.

Bulimia nervosa is an eating disorder characterized by periods of uncontrolled eating (binging) followed by compensatory behaviors (purging), most typically vomiting (>90%). Bulimia can have many adverse consequences, including severe depression, cardiac arrest, and ultimately death. Fluoxetine and other SSRIs reduce binge eating, vomiting, weight, drive for thinness, body dissatisfaction, and food and diet preoccupation. Prozac is the only SSRI approved specifically for bulimia nervosa. CBT (Cognitive Behavioral Therapy) is a form of psychotherapy that attempts to change automatic but inaccurate thoughts or beliefs in certain situations. CBT helps patients identify situations which trigger binging and purging episodes and teaches them to avoid these behaviors. There is significant unmet need for new agents that can provide: greater response rate (reduction in binging and purging); greater reduction in binging/purging in those who do respond to therapy; and/or higher abstinence rates (indicating complete elimination of binge/purge behavior).

Oral CCK-8 may be used alone in the treatment of bulimia and/or binge eating disorder and/or it may be used in conjunction with Prozac and/or CBT for treatment of obesity and/or bulimia and/or binge eating disorder; in particular embodiments the formulations to be administered are the oral formulations of the invention.

CCK-8 in an oral, enteric-coated formulation may be used for weight loss or weight control in general, and for treatment/prevention of overweight, obesity, bulimia, eating disorders, overeating, diabetes-related obesity, and metabolic syndrome, for weight loss prior to bariatric surgery, and for reduction of liver size/volume prior to any surgery, in particular bariatric surgery.

Calcitonin (see below) may be combined with CCK or analogs, in particular CCK-8, for the modulation of satiety in overweight or obese patients and for the treatment of bulimia and/or binge eating disorder. The two drugs may be in the same dosage form or may be given separately, in conjunction with each other.

Thus, there is a need for an oral CCK-8 formulation to treat the above diseases and for use in the above indications. The capsules (or tablets) comprising the oral formulation of CCK-8 may each contain 10-3000 μg CCK-8 preferably 300-1000 μg CCK-8. It is envisaged that one, two or three capsules/tablets containing CCK-8 may be self-administered prior to each meal, probably 1-3 times a day and it is possible that the CCK-8 might be administered up to four, five or six times per day.

Calcitonin: Calcitonin is a 32-amino acid linear polypeptide hormone that is produced in humans, primarily by the parafollicular cells (also known as C-cells) of the thyroid. It acts to reduce blood calcium (Ca²⁺), opposing the effects of parathyroid hormone (PTH). It has been found in fish, reptiles, birds, and other mammals. Its importance in humans has not been as well established as its importance in other animals, as its function is usually not significant in the regulation of normal calcium homeostasis.

In one aspect of the invention, calcitonin may be used alone in a dosage form in the treatment of bulimia and/or binge eating disorder and/or it may be used in conjunction with Prozac and/or CBT for treatment of bulimia and/or binge eating disorder. Calcitonin and Prozac may be in the same dosage form or may be given separately, in conjunction with each other. The dosage form may be an oral formulation, which has bioavailability. In particular the dosage form to be administered to the subject is an oral dosage form of the invention.

Vancomycin: Vancomycin is a glycopeptide antibiotic used in the prophylaxis and treatment of infections caused by Gram-positive bacteria. The original indication for vancomycin was for the treatment of methycilin-resistant Staphylococcus aureus (MRSA). Vancomycin never became first line treatment for Staphylococcus aureus, one reason being that vancomycin must be given intravenously. The prior art preparations of vancomycin need to be given intravenously for systemic therapy, since vancomycin does not cross through the intestinal lining. It is a large hydrophilic molecule which partitions poorly across the gastrointestinal mucosa. The only indication for oral vancomycin therapy is in the treatment of pseudomembranous colitis where it must be given orally to reach the site of infection in the colon. Vancomycin for use in treating or preventing infection in a subject may be administered orally to the subject by an oral formulation which has the required bioavailability. In particular, vancomycin may be formulated in accordance with one or more embodiments of the invention. Some preferred embodiments of the invention are directed to a method of treating or preventing an infection in a subject which comprises administering to the subject an oral composition, in a formulation which has the required bioavailability, wherein the therapeutic agent is vancomycin, in an amount sufficient to treat or prevent the infection. Preferably the infection is not an intestinal infection, for which existing preparations are used.

Gentamicin: Gentamicin is an aminoglycoside antibiotic, used to treat many types of bacterial infections, particularly those caused by gram-negative bacteria. When gentamicin is given orally in the prior art formulations, it is not systemically active. This is because it is not absorbed to any appreciable extent from the small intestine. Gentamicin for use in treating or preventing infection in a subject may be administered orally to the subject by an oral formulation which has the required bioavailability. In particular, gentamicin may be formulated in accordance with one or more embodiments of the invention.

In addition, compositions of the invention also can be used to treat conditions resulting from atherosclerosis and the formation of thrombi and emboli such as myocardial infarction and cerebrovascular accidents. Specifically, the compositions can be used to deliver heparin or low molecular weight heparin or fondaparinux across the mucosal epithelia.

The compositions of this invention can also be used to treat hematological diseases and deficiency states such as anemia and hypoxia that are amenable to administration of hematological growth factors. The compositions of the invention can be used to deliver vitamin B12 in a subject at high bioavailability wherein the mucosal epithelia of the subject lacks sufficient intrinsic factor. G-CSF may also be administered in accordance with various embodiments. Additionally, the compositions of this invention can be used to treat osteoporosis, such as through enteral administration of PTH, teriparatide or calcitonin once or twice or more daily.

Leuprolide (GnRH agonist) formulated in an embodiment of the invention may be delivered for treatment of female infertility (e.g., once or twice daily dosage), prostate cancer and Alzheimer's disease.

One embodiment of the invention relates to a method of treating a subject suffering from a disease or disorder which comprises administering to the subject a composition of the invention in an amount sufficient to treat the condition i.e. a therapeutically active amount. Another embodiment of the invention relates to compositions of the invention for use in treating a disease or disorder. Another embodiment of the invention relates to the use of a therapeutic agent in the manufacture of a medicament by the process of the invention for the treatment of a disorder.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter or arrest the progress of the condition. Oral dosages of the present invention, when used for the indicated effects, may be provided in the form of capsules containing 0.001, 0.0025, 0.005, 0.01, 0.025, 0.05, 0.1, 0.25, 0.5, 1.0, 2.5, 5.0, 10.0, 15.0, 25.0, 50.0 or 100, 200, 300, 400, 500, 600, 700, 800 or 1000 mg of therapeutic agent.

Compounds of the present invention may be administered in a single daily dose, or the total daily dosage may be administered in divided doses of two, three, four, five or six times daily. In some embodiments, the composition is administered at a daily dose of from about 0.01 to about 5000 mg/day, e.g., administered once daily (e.g., in the morning or before bedtime) or twice or more daily (e.g., in the morning and before bedtime). One, two, three or more capsules/tablets may be administered at each dosage time.

A representative product of the invention is an API-based formulation orally administered as an enteric coated-capsule: each capsule contains API in a solid form with Carbopol polymer and/or PVA and sodium octanoate, and suspended in a hydrophobic (lipophilic) medium containing glyceryl tricaprylate, glyceryl monocaprylate, and Tween 80; in another representative product of the invention castor oil is additionally present.

Another representative product of the invention is an API-based formulation orally administered as an enteric coated-capsule: each capsule contains API e.g., CCK-8 in a solid form with PVP and sodium octanoate and a bile salt and optionally a stabilizer, and suspended in a hydrophobic(lipophilic) medium containing: glyceryl tricaprylate and lecithin.

The compositions described herein can be administered to a subject i.e. a human or an animal, in order to treat the subject with a pharmacologically or therapeutically effective amount of a therapeutic agent described herein. The animal may be a mammal e.g., a mouse, rat, pig horse, cow or sheep. As used herein the terms “pharmacologically effective amount” or “therapeutically effective amount” or “effective amount” means that amount of a drug or pharmaceutical agent (the therapeutic agent) that will elicit the biological or medical response of a tissue, system, animal or human that is being sought by a researcher or clinician and/or halts or reduces the progress of the condition being treated or which otherwise completely or partly cures or acts palliatively on the condition.

The formulations of the invention allow incorporation of the therapeutic agent into the formulation without any chemical modification of the therapeutic agent. Additionally, as shown above, many different therapeutic agents have been successfully formulated within the formulations of the invention, including polypeptides, nucleotides, small molecules and even medium size proteins. Furthermore, the formulations of the invention allow for high flexibility in loading of the therapeutic agent. Loading capacity is dependent on the therapeutic agent. To date, loading capacity limits have not been reached; however loading of up to 1.5% wt/wt (polypeptides) and 10% wt/wt (small molecules) has been achieved and higher loading up to 33% is envisaged. Finally, the formulations of the invention protect the cargo compounds from inactivation in the GI environment due to for example proteolytic degradation and oxidation.

It is also envisaged that two or more therapeutic agents may be in the same dosage form or may be given separately, in conjunction with each other. By “in conjunction with” is meant prior to, simultaneously or subsequent to. Accordingly, the individual components of such a combination can be administered either sequentially or simultaneously from the same or separate pharmaceutical formulations. The dosage form may be an oral formulation, which has bioavailability. In particular the dosage form to be administered to the subject is an oral dosage form of the invention.

Summary of the embodiments: One aspect of the invention is a pharmaceutical composition comprising a suspension which comprises an admixture of a hydrophobic medium and a solid form wherein the solid form comprises a therapeutically effective amount of CCK-8 or an analog thereof and at least one salt of a medium chain fatty acid; this embodiment may additionally comprise a second therapeutic agent and optionally a third therapeutic agent. The pharmaceutical composition can comprise an additional constituent selected from the group consisting of a matrix forming polymer and a sugar. The solid form can comprise a particle and the particle is produced by lyophilization or by granulation or by spray-drying or can be provided as one or more dry powders or by other means. The water content in the pharmaceutical composition is lower than about 6% by weight preferably lower than about 2% by weight; the water content in the solid form is lower than about 6% by weight, preferably lower than 2% by weight. The medium chain fatty acid salt has a chain length from about 6 to about 14 carbon atoms and is sodium hexanoate, sodium heptanoate, sodium octanoate, sodium nonanoate, sodium decanoate, sodium undecanoate, sodium dodecanoate, sodium tridecanoate or sodium tetradecanoate, or a corresponding potassium or lithium or ammonium salt or a combination thereof, preferably sodium octanoate. The medium chain fatty acid salt is present in the composition at an amount of 11% to 40% by weight preferably 12% to 18% by weight, most preferably aboutl5% by weight. The medium chain fatty acid salt is present in the solid form at an amount of 50% to 90% by weight preferably at an amount of 70% to 80% by weight. The matrix forming polymer is selected from the group consisting of polyvinylpyrrolidone (PVP), cross-linked PVP (cross-povidones), ionic polysaccharides (for example, hyaluronic acid/ hyaluronates and alginic acid/ alginates), neutral polysaccharides (for example, dextran, methyl cellulose and hydroxypropyl methylcellulose (HPMC)), linear polyacrylic acid polymers including polymethacrylic acid polymers, cross-linked polyacrylic acid polymers(carbomers), amino-polysaccharides (for example chitosans), S-containing polymers(thiomers), and high molecular weight linear and bridged organic alcohols (for example, linear polyvinyl alcohol). The matrix forming polymer is present in the pharmaceutical composition at an amount of about 0.5% to 15% by weight, preferably about 1% to 10% by weight. In some embodiments the matrix forming polymer is polyvinylpyrrolidone, in particular PVP-12 and is present in the composition at an amount of about 2% to about 20% by weight, preferably at an amount of about 3% to about 18% by weight, more preferably at an amount of about 5% to about 15% by weight, most preferably at an amount of about 10% by weight. In some embodiments the cross-linked acrylic acid polymer is a sugar-cross-linked polymer, preferably a Carbopol polymer or a polyvinyl alcohol. In some embodiments the Carbopol polymer is preferably Carbopol 934P and is present in the composition at an amount of about 0.1% to about 10% by weight, preferably at an amount of about 0.5% to about 5% by weight, most preferably at an amount of about 1% by weight.

In some embodiments, the pharmaceutical compositions can additionally comprise a surfactant, which is an ionic surfactant or a non-ionic surfactant or a combination thereof. In some embodiments the surfactant is lecithin or a bile salt (e.g., sodium taurocholate) or a detergent or a combination thereof. In some embodiments the surfactant is a monoglyceride, a cremophore, a polyethylene glycol fatty alcohol ether, a sorbitan fatty acid ester, a polyoxyethylene sorbitan fatty acid ester, Solutol HS15 (polyoxyethylene esters of 12-hydroxystearic acid), an alkyl-saccharide (e.g., octyl glycoside, tetra decyl maltoside) or a poloxamer or a combination thereof. The monoglyceride can be glyceryl monocaprylate, glyceryl monoocatnoate, glyceryl monodecanoate, glyceryl monolaurate, glyceryl monomyristate, glyceryl monopalmitate or glyceryl monooleate or glyceryl monostearate or a combination thereof or wherein. In some embodiments the sorbitan fatty acid ester comprises sorbitan monolaurate, sorbitan monooleate or sorbitan monopalmitate or a combination thereof. In some embodiments the polyoxyethylene sorbitan fatty acid ester comprises polyoxyethylene sorbitan monooleate (Tween 80), polyoxyethylene sorbitan monostearate or polyoxyethylene sorbitan monopalmitate or a combination thereof. In some embodiments the surfactant is in the solid form and in some embodiments the surfactant is in the hydrophobic medium and in some embodiments the surfactant is in both the solid form and the hydrophobic medium.

In some embodiments the surfactant is lecithin or a bile salt or a detergent (e.g., Tween-80) or a combination thereof, the bile salt is sodium taurocholate, sodium deoxycholate, sodium glycocholate, sodium chenodeoxycolate, sodium cholate, sodium lithocholate, in particular sodium taurocholate. In some embodiments the hydrophobic medium comprises castor oil or glyceryl tricaprylate or glyceryl tributyrate or glyceryl monocaprylate or octanoic acid or a combination thereof. In some embodiments the main component by weight (over 50%) of the hydrophobic medium is castor oil or glyceryl tricaprylate or glyceryl monocaprylate or octanoic acid. In some embodiments the main component of the hydrophobic medium consists essentially of castor oil or glyceryl tricaprylate or glyceryl monocaprylate or octanoic acid. In some embodiments the hydrophobic medium comprises an aliphatic, olefinic, cyclic or aromatic compound, preferably an aliphatic compound, or a combination thereof. In some embodiments the hydrophobic medium comprises a mineral oil, a paraffin, a fatty acid such as octanoic acid, a monoglyceride, a diglyceride, a triglyceride, an ether or an ester, or a combination thereof. In some embodiments the ester in the hydrophobic medium is a low molecular weight ester, preferably ethyl isovalerate or butyl acetate. In some embodiments the triglyceride is a long chain triglyceride (e.g., castor oil), a medium chain triglyceride or a short chain triglyceride or a combination thereof. In some embodiments the short chain triglyceride is glyceryl tributyrate and the medium chain triglyceride is glyceryl tricaprylate or coconut oil.

In some embodiments, the pharmaceutical composition consists essentially of CCK-8, a medium chain fatty acid salt, a matrix forming polymer and a hydrophobic medium. In some embodiments the hydrophobic medium consists essentially of glyceryl tricaprylate or glyceryl monocaprylate or a combination thereof. In some embodiments the pharmaceutical composition additionally comprises a stabilizer. In some embodiments the pharmaceutical composition comprises a suspension which consists essentially of an admixture of a hydrophobic medium and a solid form, wherein the solid form comprises a therapeutically effective amount of CCK-8 or an analog thereof, at least one salt of a medium chain fatty acid and a matrix forming polymer wherein the matrix forming polymer is selected from the group comprising cross-linked acrylic acid polymer, polyvinyl alcohol polymer of molecular weight 10000-70000 Da, hyaluronic acid and salts thereof, PVP (preferably PVP-12) and cross-linked PVP (cross-povidones). The pharmaceutical composition may additionally comprise a second therapeutic agent and also a third therapeutic agent. In some embodiments the matrix forming polymer is a carbomer, the medium chain fatty acid is sodium octanoate, and the hydrophobic medium comprises glyceryl tricaprylate and one or more surfactants.

In some embodiments the carbomer is present at 0.1-3, preferably 1% by weight, the sodium octanoate is present at 10% or more by weight preferably 15%, and the surfactant is preferably present at about 6% by weight and is preferably lecithin or glyceryl monocaprylate or Tween 80 or a combination thereof. In some embodiments the matrix forming polymer is PVP, preferably PVP-12, the medium chain fatty acid is sodium octanoate, and the hydrophobic medium comprises glyceryl tricaprylate and surfactants, and optionally a stabilizer.

In one aspect of the invention, the pharmaceutical composition comprises a suspension which comprises an admixture of a hydrophobic medium and a solid form wherein the solid form comprises a therapeutically effective amount of CCK-8 or an analog thereof; sodium octanoate; and a matrix forming polymer. The pharmaceutical composition may additionally contain one or more surfactants and/or a stabilizer and/or a peptidase inhibitor. The pharmaceutical composition may additionally comprise a second therapeutic agent and also a third therapeutic agent.

In some embodiments the second or third therapeutic agent in the pharmaceutical composition is selected from the group consisting of anti-obesity or appetite suppressant drugs. The anti-obesity or appetite suppressant drug is selected from the group consisting of orlistat (Xenica™), sibutramine (Meridia™), phendimetrazine tartrate (Bontril™), methamphetamine (Desoxyn™), phentermine (Ionamin™), Adipex-P™), oxyntomodulin, an oxyntomodulin analog, PYY, PYY analog, GLP-1 and a GLP-1 analog. One embodiment is an oral dosage form comprising the pharmaceutical composition of the invention, and which is additionally enteric-coated. Another embodiment is a rectal dosage form comprising the pharmaceutical composition of the invention. One embodiment is a kit comprising instructions and the oral or rectal dosage form. One embodiment is a capsule containing the pharmaceutical composition of the invention, wherein the capsule is a hard gel or a soft gel capsule and wherein the capsule is enteric-coated.

One aspect of the invention is a method of treatment of a subject suffering from overweight (or even obese) comprising administering orally to the subject a therapeutically effective amount of CCK-8 sufficient to produce weight loss. The weight loss may be accompanied by reduction in liver size. The administration is prior to surgery, in particular bariatric surgery and the administration period is 6 months or less prior to surgery, such as 2-8 weeks prior to surgery. The method of treatment is for acute use and/or for chronic use and the administration may be prior to a meal and the administration is one, two, three, four or five times per day. The CCK-8 administration is within an enteric-coated capsule or tablet. Various embodiments comprise the method of treatment wherein the CCK-8 administered is the pharmaceutical composition of the invention, the oral dosage form of the invention and/or the capsule comprising the pharmaceutical composition of the invention. One embodiment is a method of treatment of a subject desirous of weight control comprising administering orally to the subject a therapeutically effective amount of CCK-8 sufficient to achieve weight control by the subject. Another embodiment is the pharmaceutical composition of the invention, the oral dosage form of the invention and/or the capsule comprising the pharmaceutical composition of the invention for use in treating an overweight subject in order to produce weight loss. One embodiment is a method of treatment wherein the liver size is measured before commencing of CCK-8 treatment and again just prior to surgery or wherein the liver size is measured before, during and after a period of administration of CCK-8. Further embodiments are a method of treating a subject suffering from bulimia nervosa or binge eating disorder, which comprises administering to the subject an oral composition of CCK-8 in an amount sufficient to treat the condition; and a method of stimulating gallbladder contraction in a subject which comprises administering to the subject an oral composition of CCK-8 in an amount sufficient to stimulate gallbladder contraction.

Another aspect of the invention is a process for producing a pharmaceutical composition which comprises preparing a water-soluble composition comprising a therapeutically effective amount of CCK-8 and optionally a second and optionally a third therapeutic agent, a medium chain fatty acid salt and a matrix forming polymer, drying the water soluble composition to obtain a solid powder, and suspending the solid powder in a hydrophobic medium, to produce a suspension containing in solid form the therapeutic agent, the medium chain fatty acid salt and the matrix forming polymer, thereby producing the pharmaceutical composition. Another embodiment is a process for producing a pharmaceutical composition which comprises providing a solid powder comprising a therapeutically effective amount of CCK-8 and optionally a second and optionally a third therapeutic agent, a medium chain fatty acid salt and a matrix forming polymer, and suspending the solid powder in a hydrophobic medium, to produce a suspension containing in solid form the therapeutic agent, the medium chain fatty acid salt and the matrix forming polymer, thereby producing the pharmaceutical composition. Another embodiment is a pharmaceutical composition comprising a suspension which comprises an admixture of a hydrophobic medium and a solid form wherein the solid form comprises a therapeutically effective amount of a therapeutic agent, at least one salt of a medium chain fatty acid and an additional constituent selected from the group consisting of a matrix forming polymer and a sugar; the composition may also comprise a bile salt (e.g., sodium taurocholate). In one embodiment the matrix forming polymer is selected from the group consisting of polyvinylpyrrolidone (PVP) and cross-linked PVP (cross-povidones); ionic polysaccharides (for example hyaluronic acid/hyaluronates and alginic acid/alginates); neutral polysaccharides (for example dextran, methyl cellulose and hydroxypropyl methylcellulose (HPMC)); linear polyacrylic acid polymers including polymethacrylic acid polymers; cross-linked polyacrylic acid polymers(carbomers); amino-polysaccharides (e.g., chitosans); S-containing polymers(thiomers); and high molecular weight linear and bridged organic alcohols (for example linear polyvinyl alcohol).

Another aspect of the invention is a pharmaceutical composition comprising a suspension which comprises an admixture of a hydrophobic medium and a solid form wherein the solid form comprises a therapeutically effective amount of a therapeutic agent, at least one salt of a medium chain fatty acid and a matrix forming polymer selected from the group comprising cross-linked acrylic acid polymer(carbomer), polyvinyl alcohol polymer of molecular weight 10000-70000 Da, hyaluronic acid and salts thereof, and cross-linked PVP (cross-povidones). In one embodiment the matrix forming polymer is a carbomer, preferable a Carbopol. In certain embodiments the solid form comprises a particle, and the particle is produced by lyophilization or by granulation or by spray-drying or by other means. In certain embodiments the water content in the pharmaceutical composition is lower than about 6% by weight preferably lower than about 2% by weight; and in certain embodiments the water content in the solid form is lower than about 6% by weight, preferably lower than 2% by weight. In certain embodiments the medium chain fatty acid salt has a chain length from about 6 to about 14 carbon atoms. In certain embodiments the medium chain fatty acid salt is sodium hexanoate, sodium heptanoate, sodium octanoate, sodium nonanoate, sodium decanoate, sodium undecanoate, sodium dodecanoate, sodium tridecanoate or sodium tetradecanoate, or a corresponding potassium or lithium or ammonium salt or a combination thereof, in particular sodium octanoate. In certain embodiments the medium chain fatty acid salt is present in the composition at an amount of 11% to 40% by weight preferably 12% to 18% by weight, most preferably aboutl5% by weight and in certain embodiments the medium chain fatty acid salt is present in the solid form at an amount of 50% to 90% by weight preferably at an amount of 70% to 80% by weight. In certain embodiments the matrix forming polymer is present in the composition at an amount of about 0.5% to 15% by weight, preferably about 1% to 10% by weight. In certain embodiments the matrix forming polymer is polyvinylpyrrolidone, preferably is PVP-12, and preferably has a molecular weight of about 3000, and is present in the composition at an amount of about 2% to about 20% by weight, preferably at an amount of about 5% to about 15% by weight, most preferably at an amount of about 10% by weight. In certain embodiments of the pharmaceutical composition the matrix forming polymer is Carbopol polymer, in particular Carbopol 934P. In certain embodiments the Carbopol polymer is present in the composition at an amount of about 0.1% to about 10% by weight, preferably at an amount of about 0.5% to about 5% by weight, most preferably at an amount of about 1% by weight. In certain embodiments the matrix forming polymer is polyvinyl alcohol of molecular weight of about 27000 Da, and it is present in the composition at an amount of about 0.1% to about 10% by weight, preferably at an amount of about 0.5% to about 5% by weight, most preferably at an amount of about 2% by weight. The products and processes of the invention are normally free of a medium chain alcohol and of a membrane fluidizing agent.

The pharmaceutical compositions of the invention may additionally comprise a surfactant, wherein the surfactant is in the solid form or is in the hydrophobic medium or wherein the surfactant is in both the solid form and the hydrophobic medium. The surfactant is an ionic surfactant or a non-ionic surfactant or a combination thereof. Some pharmaceutical compositions comprise a surfactant where the surfactant is lecithin or a bile salt (e.g., sodium taurocholate) or a detergent (e.g., a sorbitan fatty acid ester, Tween 80) or a combination thereof. In some compositions the surfactant is a monoglyceride, a cremophore, a polyethylene glycol fatty alcohol ether, a sorbitan fatty acid ester, a polyoxyethylene sorbitan fatty acid ester, Solutol HS15(polyoxyethylene esters of 12-hydroxystearic acid), or a poloxamer or an alkyl-saccharide or a combination thereof. In some compositions the monoglyceride is glyceryl monocaprylate, glyceryl monoocatnoate, glyceryl monodecanoate, glyceryl monolaurate, glyceryl monomyristate, glyceryl monopalmitate or glyceryl monooleate or glyceryl monostearate or a combination thereof. In some compositions the sorbitan fatty acid ester comprises sorbitan monolaurate, sorbitan monooleate or sorbitan monopalmitate or a combination thereof. In some compositions the polyoxyethylene sorbitan fatty acid ester comprises polyoxyethylene sorbitan monooleate (Tween 80), polyoxyethylene sorbitan monostearate or polyoxyethylene sorbitan monopalmitate or a combination thereof. In some compositions the alkyl-saccharide is octyl glycoside or tetra decyl maltoside or a combination thereof. In some compositions the hydrophobic medium comprises castor oil or glyceryl tricaprylate or glyceryl tributyrate or octanoic acid or a combination thereof. In some compositions 70-100% by weight of the hydrophobic medium is castor oil or glyceryl tricaprylate or glyceryl tributyrate or octanoic acid. In some compositions the main component of the hydrophobic medium consists essentially of castor oil or glyceryl tricaprylate or glyceryl tributyrate or octanoic acid. In some compositions the hydrophobic medium comprises an aliphatic, olefinic, cyclic or aromatic compound, preferably an, aliphatic compound some compositions the hydrophobic medium comprises a mineral oil, a paraffin, a fatty acid such as octanoic acid, a monoglyceride, a diglyceride, a triglyceride, an ether or an ester, or a combination thereof. The ester in the hydrophobic medium may be a low molecular weight ester, preferably ethyl isovalerate or butyl acetate. In some embodiments the triglyceride is a long chain triglyceride (e.g.,castor oil), a medium chain triglyceride (e.g., glyceryl tricaprylate or coconut oil or a combination thereof) or a short chain triglyceride (e.g., glyceryl tributyrate) or a combination thereof. In some embodiments the composition consists essentially of a therapeutic agent, a medium chain fatty acid salt and a matrix forming polymer and a hydrophobic medium. In one aspect the solid form consists essentially of a therapeutic agent, a medium chain fatty acid salt and a matrix forming polymer. In some embodiments the hydrophobic medium consists essentially of glyceryl tricaprylate or additionally contains castor oil and/or glyceryl monocaprylate and/or a surfactant. In some embodiments a pharmaceutical composition comprises a suspension which comprises an admixture of a hydrophobic medium and a solid form wherein the hydrophobic medium comprises at least one hydrophobic compound and at least one surfactant and wherein the solid form comprises a therapeutically effective amount of a therapeutic agent, at least one salt of a medium chain fatty acid, a matrix forming polymer, and optionally a stabilizer and a surfactant. In some embodiments the hydrophobic compound may be glyceryl tricaprylate and the surfactant in the hydrophobic medium may be lecithin, and the salt of a medium chain fatty acid is is sodium octanoate, the matrix forming polymer is PVP-12, and the surfactant in the solid form is a bile salt, which may be sodium taurocholate.

One aspect of the invention is a process for producing a pharmaceutical composition which comprises preparing a water-soluble solution comprising a therapeutically effective amount of at least one therapeutic agent, and optionally a second and optionally a third therapeutic agent, at least one salt of a medium chain fatty acid, a bile salt, and a constituent selected from the group consisting of a matrix forming polymer and a sugar or a combination thereof, drying the water soluble solution to obtain a solid powder, and suspending the solid powder in a hydrophobic medium comprising at least one surfactant (e.g., lecithin), to produce a suspension containing in solid form the therapeutic agent, the medium chain fatty acid salt, the bile salt, and the matrix forming polymer, thereby producing the pharmaceutical composition. In one embodiment the drying is achieved by lyophilization or by granulation or by spray-drying or by other means. One aspect of the invention is a process for producing a pharmaceutical composition which comprises providing a solid powder comprising a therapeutically effective amount of at least one therapeutic agent, and optionally a second and optionally a third therapeutic agent; at least one salt of a medium chain fatty acid; and a matrix forming polymer or a sugar or a combination thereof; and suspending the solid powder in a hydrophobic medium, to produce a suspension containing in solid form the therapeutic agent, the medium chain fatty acid salt and the matrix forming polymer or sugar; the pharmaceutical composition might additionally contain a bile salt (e.g., sodium taurocholate). In one embodiment of the process the matrix forming polymer is selected from the group consisting of polyvinylpyrrolidone (PVP) and cross-linked PVP (cross-povidones); ionic polysaccharides (for example hyaluronic acid/hyaluronates and alginic acid/alginates); neutral polysaccharides (for example dextran, methyl cellulose and hydroxypropyl methylcellulose (HPMC)); linear polyacrylic acid polymers including polymethacrylic acid polymers; cross-linked polyacrylic acid polymers(carbomers); amino-polysaccharides (e.g., chitosans); S-containing polymers(thiomers); and high molecular weight linear and bridged organic alcohols (for example linear polyvinyl alcohol) and a combination thereof. One aspect is a process for producing a pharmaceutical composition which comprises preparing a water-soluble solution comprising a therapeutically effective amount of at least one therapeutic agent, and optionally a second and optionally a third therapeutic agent at least one salt of a medium chain fatty acid, and a matrix forming polymer selected from the group comprising cross-linked acrylic acid polymer(carbomer), polyvinyl alcohol polymer of molecular weight 10000-70000 Da, hyaluronic acid and salts thereof, and cross-linked PVP (cross-povidones) or a combination thereof, drying the water soluble composition to obtain a solid powder, and suspending the solid powder in a hydrophobic medium, to produce a suspension containing in solid form the therapeutic agent, the medium chain fatty acid salt, and the matrix forming polymer, thereby producing the pharmaceutical composition. One aspect is a process for producing a pharmaceutical composition which comprises providing a solid powder comprising a therapeutically effective amount of at least one therapeutic agent, and optionally a second and optionally a third therapeutic agent; at least one salt of a medium chain fatty acid; and a matrix forming polymer selected from the group comprising cross-linked acrylic acid polymer(carbomer), polyvinyl alcohol polymer of molecular weight 10000-70000 Da, hyaluronic acid and salts thereof, cross-linked PVP (cross-povidones) and a combination thereof; suspending the solid powder in a hydrophobic medium, to produce a suspension containing in solid form the therapeutic agent, the medium chain fatty acid salt and the matrix forming polymer, thereby producing the pharmaceutical composition. In further embodiments the matrix forming polymer is a carbomer, and the carbomer is a Carbopol. One aspect is a process for producing a pharmaceutical composition which comprises preparing a water-soluble solution comprising a therapeutically effective amount of at least one therapeutic agent, at least one salt of a medium chain fatty acid, a matrix forming polymer selected from the group comprising cross-linked acrylic acid polymer (carbomer), polyvinyl alcohol polymer of molecular weight 10000-70000 Da, hyaluronic acid and salts thereof, and cross-linked PVP (cross-povidones) and a combination thereof, drying the water soluble composition to obtain a solid powder, and suspending or dissolving the solid powder in a solution consisting essentially of octanoic acid, thereby producing the pharmaceutical composition.

One aspect is a process for producing a pharmaceutical composition which comprises providing a solid powder comprising a therapeutically effective amount of at least one therapeutic agent, and optionally a second and optionally a third therapeutic agent; at least one salt of a medium chain fatty acid; and a matrix forming polymer selected from the group comprising cross-linked acrylic acid polymer(carbomer), polyvinyl alcohol polymer of molecular weight 10000-70000 Da, hyaluronic acid and salts thereof, and cross-linked PVP (cross-povidones) and a combination thereof; and suspending or dissolving the solid powder in a solution consisting essentially of octanoic acid, thereby producing the pharmaceutical composition. One aspect of the invention is the pharmaceutical composition produced by these processes. In certain embodiments of the pharmaceutical composition, the therapeutic agent is a polypeptide, a glycosaminoglycan, a polysaccharide, a small molecule or a polynucleotide; and the therapeutic agent may also be selected from the group consisting of insulin, growth hormone, parathyroid hormone or analogs thereof e.g., parathyroid hormone amino acids 1-34 termed teriparatide, interferon-alfa (IFN-α), a low molecular weight heparin, leuprolide, fondaparinux, octreotide, exenatide, terlipres sin, vancomycin, gentamicin, cholecytokinin or analogs thereof, CCK-8 and analogs thereof, calcitonin and siRNA. One aspect of the invention is an oral dosage form comprising the pharmaceutical compositions disclosed above, and the oral dosage form may be additionally enteric-coated. Another aspect of the invention is a rectal dosage form comprising the pharmaceutical compositions disclosed above and a kit comprising instructions and the oral or rectal dosage forms. Another embodiment of the invention is a capsule containing the pharmaceutical composition of the invention, wherein the capsule is a hard gel or a soft gel capsule, and wherein the capsule is enteric-coated. One aspect of the invention is a method of treating a subject suffering from acromegaly, abnormal GI motility, flushing episodes associated with carcinoid syndrome, portal hypertension, an endocrine tumor, gastroparesis, diarrhea, pancreatic leak or pancreatic pseudo-cysts which comprises administering to the subject the above compositions and formulations, wherein the therapeutic agent is octreotide, in an amount sufficient to treat the condition, disease or disorder. One aspect of the invention is a method of preventing variceal bleeding in a subject which comprises administering to the subject the above compositions and formulations, wherein the therapeutic agent is octreotide, in an amount sufficient to prevent the bleeding, in particular wherein the subject suffers from portal hypertension. One aspect of the invention is a method of treating a subject suffering from a condition, disease or disorder which comprises administering to the subject the above compositions or formulations or the oral or rectal dosage forms in an amount sufficient to treat the condition, disease or disorder.

The function and advantages of these and other embodiments will be more fully understood from the following examples. These examples are intended to be illustrative in nature and are not to be considered as limiting the scope of the systems and methods discussed herein.

### EXAMPLES

### Example 1

### Detailed Production Process of the Formulations

The production process for all the formulations described in the following Examples and throughout the specification is essentially as described in FIG. 1 and in this Example 1.

### A. Production of a Formulation of Octreotide Containing Carbopol

### Production of the Hydrophilic Fraction:

1 g of Carbopol 934P (obtained from Lubrizol) was slowly added to 200 mL water, while mixing, until complete dissolution (about 1 h). This solution was neutralized using 1.6 mL 6N NaOH solution (to pH ˜7). Upon neutralization the solution became a gel. To the gel, during mixing, 100 mL of 15% sodium octanoate solution and 5 mL of 10 mg/mL octreotide solution were added. The solution was further mixed and then lyophilized for about 24 h. This procedure produced about 18 g of hydrophilic fraction.

### Production of the Hydrophobic Medium:

2.44 g Tween 80, 4.88 g of glyceryl monocaprylate and 92.80 g of glyceryl tricaprylate were mixed together. This procedure produced about 100 g of hydrophobic medium.

### Production of the Bulk Drug Product:

Mixing of the hydrophilic fraction and the hydrophobic medium was performed using a mortar. 82.1 g of hydrophobic medium was poured into the mixing bowl. 17.9 g of the hydrophilic fraction was slowly added, while mixing. After addition of the hydrophilic fraction, the suspension was mixed for about 1 h till a smooth viscous semi-solid suspension was obtained. This resulting suspension was stored at 2-8° C. This is formulation B in Table 1A in Example 3.

### B. Production of Two Formulations of Aliskiren.

(i) A formulation of Aliskiren Containing PVP-12

### Production of the Hydrophilic Fraction

To a beaker containing 100 mL water the following ingredients were slowly added one by one (with 2-3 minutes mixing between each ingredient or until a clear solution was obtained): 10.08 g aliskiren hemifumarate (content 99.2%), 10.00 g of PVP-12 and 15.00 g of sodium octanoate. The solution was mixed for another 5 min and then transferred into stainless steel lyophilization tray. The tray was frozen at −80° C. for 1.5-2 h and then lyophilized for about 24 h. This procedure produced about 35 g of hydrophilic fraction.

### Production of the Hydrophobic Medium:

2.2 g Tween 80, 4.4 g of glyceryl monocaprylate, and 64.0 g of glyceryl tricaprylate were mixed together. This procedure produced about 70 g of hydrophobic medium.

### Production of the Bulk Drug Product:

Mixing of the hydrophilic fraction and the hydrophobic medium was performed using a mortar. 57.8 g of hydrophobic medium was poured into the mixing bowl. 32.2 g of the hydrophilic fraction was slowly added while mixing. After addition of the entire hydrophilic fraction, the suspension was mixed for about 1 h until a smooth viscous suspension was obtained, which was stored below 25° C. This is formulation I in Table 3 in Example 5.

(ii) A Formulation of Aliskiren Containing PVA (27000 Da)

The PVA of molecular weight 27000 Da was obtained from Aldrich. It was virtually completely hydrolyzed (-98-99%).

### Production of the Hydrophilic Fraction:

To a beaker containing 100 mL water, 2.00 g of PVA is added while stiffing. The mixture is heated to about 60° C. After all the PVA is dissolved, the solution is cooled to RT and 15.00 g of sodium octanoate is added while mixing. After the sodium octanoate is dissolved, 10.08 g of aliskiren hemifumarate (content 99.2%) is added while mixing. The tray is frozen at −80° C. for 1.5-2 h and then lyophilized for about 24 h. This procedure produces about 27 g of hydrophilic fraction.

### Production of the Hydrophobic Medium:

2.2 g Tween 80, 4.4 g of glyceryl monocaprylate, and 73.0 g of glyceryl tricaprylate are mixed together. This procedure produces about 80 g of hydrophobic medium.

### Production of the Bulk Drug Product:

Mixing of the hydrophilic fraction and the hydrophobic medium is performed using a mortar. 65.1 g of the hydrophobic medium is poured into the mixing bowl and 24.9 g of the hydrophilic fraction is slowly added while mixing. After addition of the entire hydrophilic fraction, the suspension is mixed for about 1 h till a smooth viscous suspension/ semi-solid is obtained. The resulting suspension is stored below 25° C. This is formulation V in Table B in Example 3.

### Example 2

### Animal Models for Testing the Bioavailability of a Range of Different Formulations Containing a Variety of Active Ingredients

In order to test the capability of the formulation platform, the bioavailability of formulations containing different cargo compounds or active ingredients (APIs) was tested in one or more of three different animal models:

(i) jejunal administration to conscious (non-anesthetized) rats;

(ii) jejunal administration to non-conscious (anesthetized) rats;

(iii) rectal administration to anesthetized rats;

These models are described below:

(i) Jejunal Administration to Conscious rats

To test the bioavailability of formulations in the jejunum of conscious rats, a specialized rat model was established in which two different cannulas are surgically implanted in male Sprague-Dowley rats (other rats may also be used):
1—jejunal cannula to bypass the stomach and enable direct formulation administration to the jejunum; and
2—jugular vein cannula to determine the systematic levels of the administered dextran following jejunal administration.

Rats were allowed to recover for 4 days before the study and were deprived of food for 18 hours before the start of the study.

Formulation containing API was administered to the jejunum of conscious rats, as described above, and separately saline solution containing API was administered intravenously or subcutaneously as reference.

Blood samples were drawn from the jugular vein at an appropriate series of times post jejunal administration and post IV administration, (or post SC administration), plasma was prepared and levels of active ingredient were determined in each sample. The average absolute Bioavailability (aBA) achieved after jejunal administration of the formulation is calculated as described below.

Exposure values, AUC (0-T), are calculated from the area under the serum concentration versus time curve (AUC) and are determined for jejunal and intravenous administration (or SC administration). T=final time at which measurement was made.

The absolute Bioavailability (aBA) is determined according to the following equation:

aBA=(jejunal AUC_{(0-T)})/(iv AUC_{(0-T)})*(iv dose/jejunal dose)

The relative Bioavailability (rBA) is determined according to the following equation

rBA=(jejunal AUC_{(0-T)}/SC AUC_{(0-T)}*(SC dose/jejunal dose)

Data is normally presented as Mean±SD (n≧5 rats per group).

(ii) Jejunal Administration to Non-Conscious (Anesthetized) Rats

The rat model described above is used. Rats are allowed to recover for 4 days after surgery, are deprived of food for 18 hours before the start of the study and are anesthetized by a solution of ketamine: xylazine.

Formulation containing API is administered via the surgically implanted cannula to the jejunum of the anesthetized rats. A saline solution containing API is administered intravenously or subcutaneously as reference.

Blood samples are drawn from the jugular vein pre-administration and at an appropriate series of times post jejunal administration and post IV administration, (or post-SC administration), plasma or serum is prepared and levels of API are determined in each sample as described in herein and/or by methods known in the art. Exposure values, AUC are determined for the formulations. The % BA is calculated compared to the exposure to the same API compound after subcutaneous or intravenous administration, as described above.

(iii) Rectal Administration to Anesthetized Rats

Male Sprague-Dowley rats (other rats may also be used) are deprived of food for 18 hours before the start of the experiment. Rats are anesthetized by a solution of ketamine: xylazine. The formulation (100 μL/rat) is administered rectally using a 14G venflon. Blood samples are drawn from the jugular vein pre-administration and at an appropriate series of times post jejunal administration and post IV administration, plasma or serum is prepared and levels of API are determined in each sample as described herein and/or by methods known in the art. The average relative Bioavailability (rBA) achieved after rectal administration of API in formulation is calculated as described above. Alternatively the API may be given IV instead of SC and the absolute Bioavailability (aBA) may be calculated as described above.

### Example 3

### Various Improved Octreotide Formulations

Various octreotide formulations were made as described in Tables 1A and 1B below. The octreotide formulation with Carbopol was prepared as described in Example 1, which produced formulation B shown below (in Table 1A). The other formulations A and C-H and J and K were prepared in essentially the same manner except instead of Carbopol, a different ingredient was substituted as shown—either PVP-12, or10% glucose, 7.5% glucose (obtained from Fluka) or PVA, MW 27K (obtained from Aldrich) or sodium alginate (obtained from Sigma) or sodium hyaluronate (obtained from Fluka) or glucosamine (obtained from Calbiochem) or polyacrylic acid (obtained from Aldrich) ; the neutralization step used for the Carbopol was omitted for most of the formulations as indicated in the Tables i.e. no NaOH added).

The formulations described above in Tables 1A and 1B (A, B, C, D, E, F, G, H, J and K) were administered directly to the jejunum of conscious rats, and plasma octreotide levels were measured post-formulation administration, essentially as described in Example 2. Exposure values, AUC, were determined for the formulations. The results are shown in Table 1A and Table 1B. These results show that all the formulations have bioavailability. The formulations which gave bioavailability similar or greater than a formulation with PVP 12 (Formulation A) were listed in Table 1A viz. formulations B (1% Carbopol 934P) and C (10% glucose) followed by D (7.5% glucose) and E (2% PVA).

The formulations which gave bioavailability less than PVP 12 were listed in Table 1B. Thus formulations containing alginate, hyaluronate, polyacrylic acid sodium salt and glucosamine in Table 1B were also active but had lower bioavailability than formulation A (PVP-12).

The Carbopol formulation was most active and in addition was a semi-solid formulation, and was not too viscous. The PVA formulation was more viscous. Note that formulations B and E had only 1% w/w or 2% w/w respectively of PVP replacement (Carbopol or PVA respectively) instead of 10% w/w of PVP in formulation A. Thus, replacing PVP 12 in the formulation by e.g., PVA or by Carbopol (or by some of the other compounds indicated) reduced the amount of matrix forming polymer in the particle phase of the formulation (the hydrophilic fraction) and thus allows more flexibility in loading of the therapeutic agent permitting increase in the amount of API that may be added; this may be desirable in order to achieve desired blood levels or reduce capsule size and number.

TABLE 1A

| | | Formulation | | | | |
|---|---|---|---|---|---|---|
| | | | 1% Carbopol | | 7.5% | 2% PVA |
| | | PVP-12 | 934P | 10% Glucose | Glucose | MW 27K |
| | | A | B | C | D | E |
| | Ingredient | (% w/w) | (% w/w) | (% w/w) | (% w/w) | (% w/w) |
| Hydrophilic | Octreotide | 0.055 = | 0.055 | 0.055 | 0.055 | 0.055 |
| fraction | powder | 0.05% or | | | | |
| | (contains 90.6% | 0.5 mg/g | | | | |
| | octreotide) | | | | | |
| | PVP 12 | 10.012 | 0 | 0 | 0 | 0 |
| | Glucose | 0 | 0 | 10.012 | 7.506 | 0 |
| | PVA (M.W 27,000) | 0 | 0 | 0 | 0 | 2.002 |
| | Carbopol 934P | 0 | 1.001 | 0 | 0 | 0 |
| | NaOH | 0 | 0.384 | 0 | 0 | 0 |
| | Sodium octanoate | 15.009 | 14.998 | 15.009 | 15.012 | 15.008 |
| | Water | 1.003 | 0.657 | 1.003 | 0.903 | 0.683 |
| Hydrophobic | Tween 80 | 2.027 | 2.018 | 2.027 | 2.080 | 2.255 |
| medium | Glyceryl | 4.036 | 4.036 | 4.036 | 4.069 | 4.491 |
| | monocaprylate | | | | | |
| | Glyceryl | 67.859 | 76.851 | 67.859 | 70.375 | 75.505 |
| | tricaprylate | | | | | |
| Results | AUC (0-25)/ | 4.2 | 5.6 | 4.3 | 4.1 | 4.5 |
| | dose/kg b.w | | | | | |
| | SD | 2.1 | 1.3 | 1.0 | 1.0 | 2.0 |
| | N | 7 | 10 | 10 | 9 | 9 |

TABLE 1B

| | | Formulation | | | | | |
|---|---|---|---|---|---|---|---|
| | | | | | | 2% Polyacrylic | 2% Polyacrylic |
| | | | | | 10% | acid sodium | acid sodium |
| | | | 1% Sodium | 2% Sodium | Glucose- | salt (average | salt (average |
| | | PVP-12 | Hyaluronate | Alginate | Amine | M.W. 1,800) | M.W. 450K) |
| | | A | F | G | H | J | K |
| | Ingredient | (% w/w) | (% w/w) | (% w/w) | (% w/w) | (% w/w) | (% w/w) |
| Hydrophilic | Octreotide | 0.055 | 0.055 | 0.055 | 0.055 | 0.055 | 0.055 |
| fraction | PVP 12 | 10.012 | 2.033 | 2.008 | 0 | 0 | 0 |
| | Sodium | 0 | 1.016 | 0 | 0 | 0.000 | 0.000 |
| | Hyaluronate | | | | | | |
| | Sodium | 0 | 0 | 2.008 | 0 | 0 | 0 |
| | Alginate | | | | | | |
| | Polyacrilyc | 0 | 0 | 0 | 0 | 2.012 | 2.012 |
| | acid | | | | | | |
| | Glucosamine | 0 | 0 | 0 | 10.006 | 0 | 0 |
| | NaOH | 0 | 0 | 0 | 0.296 | 1.006 | 1.006 |
| | Sodium | 15.009 | 15.015 | 15.002 | 15.001 | 15.013 | 15.013 |
| | octanoate | | | | | | |
| | Water | 1.003 | 0.725 | 0.763 | 1.014 | 0.253 | 0.253 |
| Hydrophobic | Tween 80 | 2.027 | 2.263 | 2.235 | 2.019 | 1.973 | 1.973 |
| medium | Glyceryl | 4.036 | 4.526 | 4.470 | 4.020 | 3.947 | 3.947 |
| | monocaprylate | | | | | | |
| | Glyceryl | 67.859 | 74.367 | 73.458 | 67.588 | 75.741 | 75.741 |
| | tricaprylate | | | | | | |
| Results | AUC (0-25)/ | 4.2 | 2.2 | 2.2 | 1.8 | 2.1 | 1.4 |
| | dose/kg b.w | | | | | | |
| | SD | 2.1 | 1.0 | 0.8 | 0.8 | 0.8 | 0.6 |
| | N | 7 | 10 | 10 | 10 | 9 | 10 |

The BA was not determined for any of the solutions in Table 1A above and Table 1B below, but only a partial AUC. However the rBA of a similar PVP-12/octreotide formulation to formulation A was determined to be 5-10%. This formulation had a higher amount of octreotide (15 mg/g instead of 0.5 mg/g) but the BA is similar when octreotide is loaded into formulations at increasing doses i.e. the amount of API in the formulation does not significantly affect BA. In other words there is linearity regarding administered dose and blood levels of API obtained (AUC=exposure).

Additional octreotide formulations were made essentially as described for Formulation B above but wherein Carbopol 934P was replaced by other Carbopols, all obtained from Lubrizol. These formulations were tested in the rat model and found to be less effective than formulations with Carbopol 934P; (they had 40-70% of the bioavailability of Formulation A. The Carbopols tested are listed below. Since determination of molecular weight of these cross-linked polymers is difficult and often inaccurate, a measure of viscosity (cP, 0.5% solution-except as indicated—at pH 7.5) is given for each Carbopol:

| | | |
|---|---|---|
| Carbopol 934P | (29,400-39,400 | cP) |
| Carbopol 971P | (4,000-11,000 | cP) |
| Carbopol 974P | (29,400-39,400 | cP) |
| Carbopol 980 | (40,000-60,000 | cP) |
| Carbopol 981 | (4,000-10,000 | cP) |
| Carbopol 5984 | (30,500-39,400 | cP) |
| Carbopol Utrez | (10-45,000-65,000 | cP) |
| Polycarbophil | (2,000-12,000 | cP, 0.2% solution) |
| Noveon AA-1 | | |

Furthermore, additional octreotide formulations were made essentially as described for Formulation E above but instead of PVA of MW 27K a range of different PVAs were used (all obtained from Aldrich). These formulations were tested in the rat model and found to be less effective than formulations with PVA of MW 27K. The PVAs tested were as follows:

M.W. 61,000 (about 80% of bioavailability of Formulation A)

M.W. 13,000-23,000, 98% hydrolysed (about 70% of bioavailability of Form. A)

M.W. 13,000-23,000,88% hydrolysed (about 35% of bioavailability of Form. A)

M.W. 10,000, 80% hydrolyzed (about 70% of bioavailability of Formulation A)

A range of PVAs is available in molecular weights up to 200K, the viscosity of formulations increasing with increase of molecular weight. Experimental work with octreotide formulations demonstrated that PVA of lower MW (27000) performed better than higher (61000), and thus PVAs of lower MWs were then tested. PVA may be partially or complete hydrolyzed being produced by synthesis from polyvinyl acetate. The PVAs checked including 27000 were virtually completely hydrolyzed (˜98-99%), except as indicated.

### Example 4

### A Selection of Compounds in the Carbopol Formulation

A range of formulations containing Carbopol 934P were prepared essentially as described for Formulation B above. These formulations contained a variety of cargo compounds: FD4 (obtained from Sigma) insulin (obtained from Dyosynth) , gentamicin (obtained from Applichem), vancomycin (obtained from Gold Biotechnology), exenatide (obtained from Novetide) and CCK-8 (obtained from Apollo). The FD4 15 FITC-labeled dextran with an average MW of 4.4 kDa, FD4). A formulation containing siRNA was also devised. These formulations are shown in Table 2.

TABLE 2

| | | Formulation | | | | | | |
|---|---|---|---|---|---|---|---|---|
| | | FD4 | Insulin | Gentamicin | Vancomycin | Exenatide | CCK8 | siRNA |
| | Ingredient | (% w/w) | (% w/w) | (% w/w) | (% w/w) | (% w/w) | (% w/w) | (% w/w) |
| Hydrophilic | API | 0.545 | 0.488 | 2.169 | 2.169 | 0.055 | 0.050 | 5.102 |
| fraction | Carbopol | 1.000 | 1.000 | 1.000 | 1.000 | 1.000 | 1.000 | 1.000 |
| | 934P | | | | | | | |
| | Sodium | 15.000 | 15.000 | 15.000 | 15.000 | 15.000 | 15.000 | 15.000 |
| | octanoate | | | | | | | |
| | NaOH | 0.360 | 0.368 | 0.367 | 0.360 | 0.360 | 0.360 | 0.360 |
| | Water | 1.033 | 1.033 | 1.003 | 1.003 | 1.033 | 1.033 | 0.858 |
| Hydrophobic | Tween 80 | 2.000 | 2.000 | 2.000 | 2.000 | 2.000 | 2.000 | 2.000 |
| medium | Glyceryl | 4.000 | 4.000 | 4.000 | 4.000 | 4.000 | 4.000 | 4.000 |
| | monocaprylate | | | | | | | |
| | Glyceryl | 76.062 | 76.111 | 74.431 | 74.431 | 76.552 | 76.557 | 71.68 |
| | tricaprylate | | | | | | | |

The formulation containing FD4 was tested as described in Example 2, and the absolute BA for FD4 was determined to be 12.5% (SD=5.7; N=10). A formulation similar to Formulation A with FD4 from the same lot gave BA of 12.0%±7.2%, N=6.

The other formulations are to be tested for bioavailability in the animal model described in Example 2, namely, by jejunal administration to conscious (non-anesthetized) rats. The bioavailability for each formulation is determined as described. CCK-8 is measured by ELISA and LC/MS/MS.

### Example 5

### Influence of Bile Salts on Bioavailability of Octreotide

It was decided to evaluate the effect of the bile salts on bioavailability of octreotide. The bile salt investigated were sodium taurocholate, sodium chenodeoxycholate and sodium deoxycholate. Formulations were prepared based on the octreotide Formulation A in Table 1A above, except that there was 0.75% octreotide and additionally the hydrophilic fraction contained one of these three bile salts at 1% of the total formulation (% w/w). Rats (conscious) were administered formulation through the jejunal cannula as described in Example 2. Octreotide PK in blood was evaluated following administration of the formulations and compared to octreotide PK following administration as control of
Formulation A (0.75% octreotide).
Formulation A: BA=4.7% (58% CV, n=6)
Formulation A plus sodium taurocholate: BA=9.2% (44% CV, n=6)
Formulation A plus sodium deoxycholate: BA=5.1% (37% CV, n=5)
Formulation A plus sodium chenodeoxycholate: BA=6.8% (38% CV, n=6)

These results showed that 1% sodium taurocholate improved octreotide bioavailability about two-fold compared to Formulation A. These results also indicated that ,at a level of 1%, the unconjugated bile salts deoxycholate and chenodeoxycholate showed no significant improvement on octreotide bioavailability, although chenodeoxycholate perhaps showed a trend of enhanced bioavailability.

### Example 6

### Formulations for Aliskiren

Various formulations of aliskiren (Novartis) were devised as described in Table 3 below, and all five formulations were prepared. The formulations were prepared essentially as described in Example 1, wherein the ingredients in the hydrophilic fraction and in the hydrophobic medium for each formulation were as listed in Table 3. The neutralization step was used only for the Carbopol formulation (Formulation II) and was omitted from the other formulations. Each formulation contains 10% of aliskiren free base.

TABLE 3

| | | Formulation | | | | |
|---|---|---|---|---|---|---|
| | | | 1% Carbopol | | Octanoic | 1% PVA |
| | | PVP12 | 934P | Basic | acid | 27000 Da |
| | | (I) | (II) | (III) | (IV) | (V) |
| | Ingredient | (% w/w) | (% w/w) | (% w/w) | (% w/w) | (% w/w) |
| Hydrophilic | Aliskiren | 10.0 | 10.0 | 10.0 | 10.0 | 10.0 |
| fraction | PVP 12 | 10.0 | 0 | 2.75 | 10.0 | 0 |
| | PVA (M.W 27,000) | 0 | 0 | 0 | 0 | 1.0 |
| | Carbopol 934P | 0 | 1.0 | 0 | 0 | 0 |
| | NaOH | 0 | 0.384 | 0 | 0 | 0 |
| | Sodium octanoate | 15.0 | 15.0 | 12.0 | 15.0 | 15.0 |
| | Water | 0.711 | 0.538 | 0.506 | 0.702 | 0.522 |
| Hydrophobic | Tween 80 | 2.0 | 2.0 | 0 | 0 | 2.0 |
| (lipophilic) | Glyceryl mono- | 4.0 | 4.0 | 0 | 0 | 4.0 |
| medium | caprylate (GMC) | | | | | |
| | Glyceryl tricaprylate | 58.2 | 67.00 | 0 | 0 | 67.4 |
| | (GTC) | | | | | |
| | Span40 | 0 | 0 | 1.2 | 0 | 0 |
| | Lutrol F-68 | | | | 6.0 | |
| | Lecithin | 0 | 0 | 2.4 | 0 | 0 |
| | Ethyl-iso-valerate | 0 | 0 | 10.0 | 0 | 0 |
| | Glyceryl mono-oleate | 0 | 0 | 2.3 | 0 | 0 |
| | Glyceryl tributyrate | 0 | 0 | 19.6 | 0 | 0 |
| | Castor oil | 0 | 0 | 39.2 | 0 | 0 |
| | Octanoic acid | 0 | 0 | 0 | 58.2 | 0 |
| Results | AUC_{(0-240 min)}/dose | 2188 | 1429 | 1938 | 2182 | 1667 |
| | (ng*min/mL)/(mg/kg) | | | | | |
| | CV, % | 65 | 63 | 39 | 49 | 68 |
| | N | 10 | 10 | 9 | 10 | 10 |
| | Fold from Aliskiren | 2.2 | 1.4 | 2.0 | 2.2 | 1.7 |
| | oral solution | | | | | |

In Formulation I, a major constituent of the hydrophilic fraction is PVP. The significance of PVP as a structuring element was examined by replacing it with different matrix forming polymers, namely, Carbopol and polyvinyl alcohol (27 kDa PVA) and Formulations II and V respectively. Formulation IV, based on octanoic acid, was made as described for the other four formulations see e.g., FIG. 1 and appears to be a semi-transparent liquid, but still a suspension.

The above formulations in Table 3 were tested for bioavailability in the animal model described in Example 2(i) namely, by jejunal administration to conscious (non-anesthetized) rats. Aliskiren was measured using LC/MS/MS and the bioavailability for each formulation was determined as described. This was then compared with the bioavailability of the unformulated oral aliskiren solution given by gavage, which gave the following results: AUC_{(0-240 min)}/dose/kg=990, CV=53%, N=10.

These results are shown in the above Table 3. All the formulations tested showed an increase in bioavailability compared to an unformulated oral solution of aliskiren. Additionally, the 10% PVP formulation, Formulation I, was prepared in two versions, the lyophilized version described above, and a non-lyophilized version I (formulation I-NL) which gave the following results: AUC_{(0-240 min)}/dose/kg=1654, CV=122%, N=9, fold from Aliskiren oral solution=1.7

Stability data: Three of the above formulations in Table 3, and also Formulation I-NL were tested for stability by incubation at 25° C. for two months. The identification of individual impurities and degradants was performed using HPLC. The total IDD results (Impurities and Degradants Determination) were as follows: Formulation I=4.57%, Formulation III=7.29%, Formulation IV=35.37%, and Formulation I-NL lost homogeneity after two weeks and could not be measured.

Formulation I was chosen as the candidate on which to base future development, based on stability and bioavailability data

### Example 7

### Variation of Amount of Permeability Enhancer and PVP

Permeability enhancer: Medium chain fatty acid salts are key excipients for increased absorption in the formulations of the invention, and in particular sodium octanoate (sodium caprylate) is used. Earlier formulation work done by the inventors showed that bioavailability increased in a linear relationship with the concentration of sodium octanoate (from 3 to 12%). When further increase in sodium octanoate was tested (12, 15 and 18% sodium octanoate) the results clearly demonstrated highest bioavailability at 15% sodium octanoate concentration. All these experiments were performed with different APIs, wherein significantly lower loading was needed (about 0.05-0.1% compared to about 10% for aliskiren). The assumption was that probably higher permeability enhancer concentration might be needed for these levels of loading.

In order to verify this, Formulation IA was made, which had increased sodium octanoate concentration (20%) and reduced PVP concentration (2.75%) in order “to make room” for the sodium octanoate ; this formulation resulted in reduction of about 30% in bioavailability compared to Formulation I, as shown in Table 4.

TABLE 4

| | AUC_{(0-240 min)}/ | | |
|---|---|---|---|
| Formulation | dose/kg (CV) | N | Fold from gavage |
| Aliskiren oral solution | 990 | 10 | 1 |
| (gavage) | (53%) | | |
| Formulation I | 2188 | 10 | 2.2 |
| (see Table 3) | (65%) | | |
| Formulation IA- | 1427 | 9 | 1.4 |
| containing | (63%) | | |
| 20% sodium octanoate, | | | |
| 2.75% PVP | | | |

PVP: Another consideration was regarding the level of PVP. The API loading must be maintained relatively high in order to achieve therapeutic aliskiren blood concentrations. Thus it could be helpful to try to reduce the level of the matrix forming polymer in order to get more API loading capacity and reduce viscosity of the formulations. This was done with the non-lyophilized (NL) version of Formulation I, Formulation I-NL as shown in Table 5. Formulation I-NL-3% PVP is similar to Formulation I-NL except for reduction in amount of PVP to 3%.

TABLE 5

| | AUC_{(0-240 min)}/ | | |
|---|---|---|---|
| Formulation | dose/kg (CV) | N | Fold from gavage |
| Aliskiren oral solution | 990 | 10 | 1 |
| (gavage) | (53%) | | |
| Formulation I-NL | 1654 | 9 | 1.7 |
| | (122%) | | |
| Formulation I-NL-3% | 1195 | 9 | 1.2 |
| PVP | (51%) | | |

As shown in Table 5, the reduction in the amount of PVP resulted in a slight decrease in bioavailability and was therefore not implemented in future formulations.

### Example 8

### Investigation of Polar Compounds to Replace Octanoic Acid

The octanoic acid based formulation (Formulation IV in Table 3) displayed good bioavailability and prolonged PK profile (which is preferable), but also produced 35.7% total degradants after incubation at 25° C. for two months, as measured by HPLC. In order to try to preserve the bioavailability advantages and improve stability, different polar lipophilic compounds were checked, which were less acidic and less polar than octanoic acid. These lipophilic compounds were glyceryl monocaprylate (GMC), ethyl octanoate, Captex 200 (propylene glycol dicaprylate/dicaprate) and Poloxamers 123, 124 and 181. The formulations were prepared based on Formulation I in Table B wherein the lipophilic(hydrophobic) phase was replaced by one of the compounds above. The formulations were checked for stability and/or bioavailability. Results are shown in Table 6 below.

TABLE 6

| | AUC_{(0-240 min)}/ | | |
|---|---|---|---|
| Formulation | dose/kg (CV) | N | Fold from gavage |
| Aliskiren oral solution | 990 | 10 | 1 |
| (gavage) | (53%) | | |
| Formulation I | 2188 | 10 | 2.2 |
| | (65%) | | |
| Formulation IV | 2182 | 10 | 2.2 |
| | (49%) | | |
| Formulation IV-NL, with | 3855 | 19 | 3.9 |
| GMC as lipid phase | (126%) | | |
| Formulation I, | 1515 | 10 | 1.5 |
| with Captex 200 | (101%) | | |
| (0.5% NaTC, 6% Lecithin) | | | |

These results show that using GMC as lipid phase gives good bioavailability. Formulations which contained other polar lipophilic compounds were not checked for bioavailability either because there was a lack of stability improvement (ethyl octanoate, Poloxamer 124) or because the formulations were toxic to rats (Poloxamers 123 and 181).

### Example 9

### Effect of Different Surfactants on Bioavailability and Stability of the Formulations

Most of the formulas of the invention are a suspension of hydrophilic particles in a lipophilic(hydrophobic) medium. When the formulation is placed into the GI environment, it is exposed to an aqueous medium and an emulsion presumably will form. The properties of the emulsion, including the absorption ability, should be strongly linked to the properties of the surfactant blend. Several experiments were designed in order to optimize the surfactants in order to produce higher bioavailability/stability.

HLB approach. Experiments were performed to optimize the HLB of the surfactants blend using different ratios of lipophilic and hydrophilic surfactants. A combination of Labrafil (HLB 4) and Lutrol F-68 (HLB 29) was used (at 6% overall surfactant concentration) and covered the HLB range from 5.3 to 27.8. These experiments resulted in formulations which were too viscous and had reduced bioavailability.

Bile mimicking. A series of formulations was prepared, based on Formulation I (in Table 3) with 6% lecithin as the single surfactant (replacing GMC and Tween 80) in the hydrophobic medium and with addition of various amounts of sodium taurocholate (Na-TC) to the solid (hydrophilic) phase. The formulations were checked for bioavailability as shown in Table 7 below.

TABLE 7

| | AUC_{(0-240 min)}/ | | |
|---|---|---|---|
| Formulation | dose/kg (CV) | N | Fold from gavage |
| Aliskiren oral solution | 990 | 10 | 1 |
| (gavage) control | (53%) | | |
| Formulation I | 2188 | 10 | 2.2 |
| | (65%) | | |
| Formulation I including | 2497 | 9 | 2.5 |
| 6% Lecithin | (45%) | | |
| Formulation I including | 3347 | 9 | 3.4 |
| 0.1% Na-TC, 6% lecithin | (150%) | | |
| Formulation I including | 6897 | 10 | 7.0 |
| 0.5% Na-TC, 6% lecithin | (78%) | | |
| (now designated | | | |
| Formulation VI) | | | |
| Formulation I including | 2217 | 10 | 2.2 |
| 1% Na-TC, 6% lecithin | (62) | | |
| Formulation I including | 2813 | | |
| 3% Na-TC, 6% lecithin | (91) | 10 | 2.8 |

Table 7 shows dose-response results for sodium taurocholate (designated Na-TC), and demonstrates that incorporation into the formulations of a bile component (sodium taurocholate) produces an improvement in bioavailability, with a maximum effect at 0.5% sodium taurocholate. Formulation I with 0.5% Na-TC in the solid (hydrophilic) phase and 6% lecithin as the single surfactant is designated Formulation VI ; see Table 8 in Example 10 for its detailed composition. A similar sodium taurocholate dose-response experiment was performed using 2% aliskiren in a similar series of formulations but without lecithin; this experiment also demonstrated that a formulation comprising 0.5% Na-TC in the solid (hydrophilic) phase gave the highest bioavailability, in this case a 5.6 fold increase over gavage.

The stability of two of the formulations in Table 7, Formulation I and Formulation VI (having 0.5% Na-TC, 6% lecithin) was then investigated. The formulations were maintained at 25° C. for 4 weeks and at 40° C. for 4 weeks and analyzed for degradants. The results of the two formulations were very similar and demonstrated that incorporation of lecithin and sodium taurocholate had no significant effect on stability.

Other surfactants: Surfactants that are blends of several components and include PEG-based were investigated (e.g., labrasol). The formulations were found not to be stable. Incorporation of Lutrols (F-68, F-127) produced formulations with inferior bioavailability compare to 6% lecithin.

Thus the formulation with the best bioavailability was modified Formulation I, containing 0.5% sodium taurocholate in the solid form and 6% lecithin as sole surfactant in the hydrophobic medium, and this formulation was designated Formulation VI; see detailed composition of this formulation in Table 10, in Example 12.

### Example 10

### Stability of the Aliskiren Formulations

Stability of the aliskiren was one of the major concerns in formulation development. The three most probable reasons for degradant formation were investigated: a) residual water content in the excipient; b) residual acid content in the excipients; and c) metal catalysis:

a) Residual water: The water content of the hydrophilic fraction (HFP) of the formulations is controlled and does not exceed 2%. The only possibility to have additional residual water in the formulation is the water content of the lipidic excipients. Several excipients (GMC, GTC, Tween-80 and octanoic acid) were checked for water content by KF titration, and the stability of aliskiren in each excipient was measured at 25° C. and 40° C. at intervals up to 2 months. It was found that there was no correlation between water content in the excipient to aliskiren stability in the corresponding excipient.

b) Residual acidity: The correlation between the acid values of several excipients to the stability results was examined. Aliskiren was incubated separately with one of four excipients (octanoic acid, GMC, Tween-80 and GTC) having decreasing acid value, respectively, at 25° C. and at 40° C. for up to two months, and the amount of impurities and degradants was measured. It was found that there indeed was a positive correlation between the acidity of the excipient to the destabilization produced by the excipient. In other words, the most acid medium, octanoic acid, produced the most impurities and degradants, while the least acid medium, GTC, produced the least impurities and degradants.

To investigate this point, two GTC based formulations were prepared. This was Formulation I without surfactants and with/without addition of 2% acetic acid. The stability results at 25° C. for 4 weeks showed that addition of acidity to the formulation caused significant increase in degradation. Attempts were made to solve this problem by buffering the system, and different bases (organic amines) were added to aliskiren dispersion into GMC (imidazole, niacinamide and N,N-Diisopropylethylamine) or to Formulation I (niacinamide or ethyl-nicotinate, either to hydrophilic fraction or lipophilic medium or both) without any noticeable stabilization effect. Apparently, residual acidity is not a major degradation source in this type of formulation.

c) Metal catalysis. The effect of residual metals on aliskiren stability was studied. In order to check the quantity of residual heavy metals, Inductively Coupled Plasma (ICP) analysis of the ingredients in Formulation I was performed; these ingredients are sodium octanoate, PVP-12, Tween 80, GMC and GTC. The only suspicious finding was a high boron level found in sodium octanoate. Boron was therefore investigated as a possible aliskiren destabilizer. It was found, however, that borate at different pH values did not affect the aliskiren stability in solution.

### Example 11

### Additional Approaches to Produce Stability of the Aliskiren Formulations

Following the lack of success of the above investigation (Example 10), the inventors attempted to solve the stability problem in the following ways:

(i) Antioxidants. Several antioxidants were tried in the early stage of the development with GMC based formulations. Antioxidants incorporated in the hydrophilic fraction (0.025% Torlox) or dispersed in the lipophilic medium (0.03% α-tocopherol, 0.02% BHA) had no stabilizing effect.

(ii) Replacing GMC by GTC. GMC-based formulations showed the best bioavailability but limited stability. GTC-based formulations having GTC as the sole ingredient of the lipophilic phase were made and tested for stability by incubation at 25° C. and at 40° C. for two weeks, The identification of individual impurities and degradants was performed using HPLC. The total IDD results (Impurities and Degradants Determination) were as follows: After incubation at 25° C. for two weeks Formulation I with GTC only as lipophilic phase had IDD=1.12% and Formulation I with GMC only as lipophilic phase had IDD=1.74%. After incubation at 40° C. for two weeks, Formulation I with GTC only as lipophilic phase had IDD=3.04% and Formulation I with GMC only as lipophilic phase had IDD=7.01%.

(iii) Polar Non Hydroxylic Substitutes for GTC in the Lipophilic(Hydrophobic) Medium

GMC based formulations of the invention were shown to have the best bioavailability but limited stability, probably because of the high content of hydroxides. Therefore, it was decided to try to identify a polar, continuous phase that would give similar bioavailability without harming stability. Ethyl octanoate, Captex 200 (propylene glycol dicaprylate/dicaprate) and Poloxamers 123 and 181 were all investigated as the lipid phase of formulations of the invention. There was no improvement in stability results in all three formulations. Moreover, there was no improvement in bioavailability i.e. the bioavailability was less than the corresponding formulation with GMC (see for example Table 6 above).

(iv) Metal salts. Different salts of divalent cations (magnesium, calcium and zinc compounds) were incorporated into the hydrophilic fraction of Formulation I (lipophilic medium GTC only) and of Formulation VI; different salt concentrations (1:1 or 1:2 molar ratio of aliskiren: stabilizer) were tested. They were then tested for stability by incubation at 25° for four weeks and at 40° for two weeks. The identification of individual impurities and degradants was performed using HPLC. The total IDD results (Impurities and Degradants Determination) were as follows, for 25° and for 40°, respectively: Formulation I alone=2.00% and 2.80%; Formulation I plus 3.0% zinc acetate=0.74% and 1.95%; Formulation I plus 1.6% magnesium chloride=1.30% and 4.76%; Formulation VI plus 1.6% magnesium chloride=0.9% and 6.12%; Formulation VI plus 2.23% zinc chloride=1.17% and 5.59%; Formulation VI plus 4.47% zinc chloride=0.90% and 2.71%; Formulation VI plus 1.82% calcium chloride=1.23% and 4.96%; Formulation VI plus 3.0% zinc acetate=0.50% and 2.21%; and Formulation VI plus 6.0% zinc acetate=0.30% and 1.92%. Addition of the following salts was also examined: ferrous acetate, ferric citrate and sodium acetate. There was no improvement in stability. Thus formulations containing zinc acetate showed the best stability. Following the encouraging stability results, several formulations of the above formulations were checked for bioavailability, and some of the results are shown in Table 8 below.

TABLE 8

| | AUC_{(0-240 min)}/ | | |
|---|---|---|---|
| Formulation | dose/kg (CV) | N | Fold from gavage |
| Aliskiren oral solution | 990 | 10 | 1 |
| (gavage) control | (53%) | | |
| Formulation I | 2188 | 10 | 2.2 |
| | (65%) | | |
| Formulation VI | 6897 | 10 | 7.0 |
| | (78%) | | |
| Formulation VI, plus | 1051 | 10 | 1.1 |
| 2.59% CaCl₂ | (69%) | | |
| Formulation VI, plus | 1925 | | |
| 3.0% ZnAc₂ | (62%) | 10 | 2.0 |

(v) Amino acids. The inventors postulated that amino acids (e.g., glycine, aspartic acid and arginine) might be used as stabilizers. Glycine (1.23%), arginine (2.86%) and aspartic acid (2.2%), were each added separately to the hydrophilic fraction of Formulation VI. These formulations and also Formulation VI without amino acid were tested for stability by incubation at 25° C. for one week and at 40° C. for one week. The identification of individual impurities and degradants was performed using HPLC. The total IDD results (Impurities and Degradants Determination) were as follows for 25° and for 40° respectively: Formulation VI alone=0.62% and 3.55%; Formulation VI plus glycine=0.44% and 2.93% ; Formulation VI plus arginine=0.36% and 1.59%; and Formulation VI plus aspartic acid=0.66% and 5.02%. Guanidine was also tested but had no significant effect on stability.

Based on these results, higher arginine concentrations in Formulation VI were investigated for stability. The total IDD results at 25° C. for 4 weeks and at 40° C. for two weeks, respectively, were as follows: Formulation VI alone=0.90% and 6.12%; Formulation VI plus 2.86% arginine=1.07% and 3.57%; Formulation VI plus 5.72% arginine=1.00% and 3.27%. Arginine-containing formulations of the invention were then tested for bioavailability, and the results are shown in Table 9.

TABLE 9

| | AUC_{(0-240 min)}/ | | |
|---|---|---|---|
| Formulation | dose/kg (CV) | N | Fold from gavage |
| Aliskiren oral solution | 990 | 10 | 1 |
| (gavage) control | (53%) | | |
| Formulation I | 2188 | 10 | 2.2 |
| | (65%) | | |
| Formulation VI | 6897 | 10 | 7.0 |
| | (78%) | | |
| Formulation VI, plus | 4448 | 10 | 4.5 |
| 2.86% Arg | (69%) | | |
| Formulation VI, plus | 2985 | 10 | 3.0 |
| 5.72% Arg | (23%) | | |

As the results in Table 9 above demonstrate, the formulations containing arginine have bioavailability.

(vi) Combination formulations. Combination formulations are formulations including both metal salts and amino acids. Since arginine-containing formulations and ZnAc₂ containing formulations (above) show significant decrease in degradant formation, it was decided to try to make combination formulations. Making the hydrophilic fraction including both Zn acetate and arginine was found to be problematic since sticky sediment formed during manufacture. Finally, a combination Zn acetate/arginine formulation was made which contained 3% Zn acetate/ 2.86% arginine, and it was prepared using two different hydrophilic fractions:
1^{st} hydrophilic fraction: Half the amount of aliskiren, half the amount of sodium taurocholate, all the PVP and all the Zn acetate were dissolved in half the amount of water, frozen and lyophilized.
2^{nd} hydrophilic fraction: Half the amount of aliskiren, half the amount of sodium taurocholate, all the sodium octanoate and all the arginine were dissolved in half the amount of water, frozen and lyophilized

After lyophilization, both hydrophilic fractions were mixed together into the lipophilic medium to produce one bulk drug product.

The stability results were as follows: Incubation at 25° C. for 3 months, IDD=0.40%; incubation at 40° C. for 4weeks, IDD=0.79%; incubation at 4° C. for 2 month, IDD=0.08%. Thus this combination formulation is very stable, but on testing in the animal model this formulation was found to have low bioavailability, and showed no improvement over gavage. Other combinations were made with arginine: magnesium acetate (using two different concentrations of both arginine and magnesium acetate), and calcium acetate. Other metal salts did not form the precipitation seen with zinc salts and could be made in the regular manner (producing one hydrophilic fraction), but a combination of other metal salts with arginine did not give such marked stability improvement as the combination formulation of zinc acetate/arginine.

(vii) Other approaches tested. Several additional approaches were tried in order to improve stability of the formulations. These approaches included: dry granulation instead of lyophilization (to reduce exposure of the aliskiren to water) and use of glycerol (addition as stabilizer to HFP or to LFP, or coating of HPF powder with glycerol before mixing with LFP). All these efforts did not result in any noticeable stabilization effect.

(iix) Temperature effect. During the stability program, it was noticed that the degradation level at 40° C. is not in line with the stability results at 25° C. It seems that degradation at 40° C. is much higher than might be expected. Therefore, several formulations were investigated to explore the effect of incubation at 2-8° C. The results clearly indicated, that in each single formulation tested at refrigerator temperatures (2-8° C.), no significant degradants formed even after 2 months of storage. The results of stability experiments at 4° C. are as follows:
Formulation VI, IDD=0.15% at 4 weeks
Formulation VI-Zn (Formulation VI with 3.0% zinc acetate), IDD=0.10% at 2 months
Formulation VI-Arg (Formulation VI with 5.72% arginine), IDD=0.67% at 2 months

### Example 12

### Three Aliskiren Formulations

Based on a combination of bioavailability and stability results, three key formulations were noted:
Formulation VI: This was derived from Formulation I, with 0.5% sodium taurocholate in the hydrophilic fraction and 6% lecithin instead of Tween/GMC in the lipophilic medium.
Formulation VI-Zn: Formulation VI with 3.0% zinc acetate as stabilizer
Formulation VI-Arg: Formulation VI with 5.72% arginine as stabilizer.

For clarity, the composition of these three formulations is as shown in Table 10.

TABLE 10

| | | Formulation # | | |
|---|---|---|---|---|
| | | VI | VI-Zn | VI-Arg |
| | | TCL | 3% Zn | 5.72 Arg % |
| | Ingredient | (% w/w) | (% w/w) | (% w/w) |
| Hydro- | Aliskiren, as | 10 | 10 | 10 |
| philic | hemifumarate | | | |
| fraction | PVP 12 | 10 | 10 | 10 |
| | Sodium octanoate | 15 | 15 | 15 |
| | Sodium taurocholate | 0.5 | 0.5 | 0.5 |
| | Zinc acetate | — | 3.0 | — |
| | Arginine | — | — | 5.72 |
| | Residual water | 0.7 | 0.8 | 0.8 |
| | (assumed 2% | | | |
| Hydro- | Lecithin | 6.0 | 6.0 | 6.0 |
| phobic | Glyceryl tricaprylate | 57.7 | 54.6 | 51.9 |
| (lipophilic) | (GTC) | | | |
| medium | | | | |
| Results | AUC_{(0-240 min)}/ | 6897 | 1925 | 2985 |
| | dose/kg b.w | | | |
| | CV | 78 | 62 | 23 |
| | N | 10 | 10 | 10 |
| | Fold from gavage | 7.0 | 2.0 | 3.0 |

### Example 13

### Additional Formulations for CCK-8

Various formulations of CCK-8 (Sincalide, obtained from Apollo) were devised as described in Table 11 below, and Formulations R, S and T were prepared. The formulations were prepared essentially as described in Example 1, wherein the ingredients in the hydrophilic fraction and in the hydrophobic(lipophilic) medium for each formulation are as listed in Table 11, and where the amount of CCK-8 varied as described below.

TABLE 11

| | | Formulation | | | |
|---|---|---|---|---|---|
| | | | | GMC (Non | Taurocholate/ |
| | | Basic | GTC | lyophilized) | lecithin |
| Ingredient % w/w | | Q | R | S | T |
| Hydro- | CCK-8 | 0.051 | 0.051 | 0.051 | 0.051 |
| philic | PVP-12 | 2.75 | 10.0 | 10.0 | 10.0 |
| fraction | Sodium octanoate | 12.0 | 15.0 | 15.0 | 15.0 |
| | Sodium taurocholate | — | — | — | 0.50 |
| | Water | 0.592 | 1.00 | — | 1.02 |
| Hydro- | Span40 | 1.2 | — | — | — |
| phobic | Lecithin | 2.4 | — | — | 6.0 |
| (lipophilic) | Ethyl-iso-valerate | 10.0 | — | — | — |
| medium | Glyceryl monooleate | 2.30 | — | — | — |
| | Glyceryl tributyrate | 22.9 | — | — | — |
| | Castor oil | 45.8 | — | — | — |
| | Tween 80 | — | 2.0 | — | — |
| | Glyceryl Monocaprylate | — | 4.0 | 74.95 | — |
| | Glyceryl Tricaprylate | — | 67.95 | — | 67.43 |

Three of the above formulations (R, S and T) were prepared as follows for use in testing biological activity: Formulation R at levels of 0.165, 0.055, 0.003 mg CCK-8 per rat; Formulation S at 0.09 mg CCK-8 per rat; and Formulation T at 0.09 mg CCK-8 per rat. Thus Formulation R with 0.055 CCK-8 (% w/w) was used at 0.3 and 0.1 ml per rat to give a dose of 0.165 and 0.055 (mg per rat); Formulation R with 0.001 CCK-8 (% w/w) was used at 0.3 ml per rat to give a dose of 0.003 mg per rat; and Formulations S and T with 0.03 CCK-8 (% w/w) were both used at 0.3 ml to give a dose of 0.09 mg per rat.

The effect of oral administration of CCK8 on food intake was achieved by measuring the volume of D-glucose consumed by conscious rats (essentially using the method described by Chun-Min et al., Am. Physio. Soc. 296:R43-R50, 2009). The feeding response of conscious rats to exogenous oral CCK-8 was analyzed by measuring the volume of D-glucose solution consumed. Sprague Dawley rats cannulated at the jejunum (as described in Example 2) were fasted for 18 h. The fasted animals were administrated with a dose range of exogenous CCK8 using three formulations. Three groups served as controls: Rats administered with formulation alone; rats administered with CCK-8 in saline (0.165 mg

CCK-8 per rat); and rats that did not receive any treatment (no treatment). Five minutes post administration the rats were given access to a solution of 0.125 g/ml D-glucose in water. The volume of glucose solution consumption was measured at 30 min, 60 min and 90 min post administration. Analysis of glucose consumption for the different treatment groups showed a significant effect at 30 min post administration, as presented in Table 12 below. Analysis of median values and average values of the three control groups, i.e. formulation alone, no treatment and CCK8 in saline, showed similar levels (P>0.05). However, there was significant inhibition of food intake in the rats which received formulation R at 0.165 mg and 0.055 mg CCK-8 per rat, and also in the rats which received formulation S and formulation T (both 0.09 mg CCK-8 per rat), compared to the control groups. The effect of the formulated CCK8 on food intake was further supported by a dose-related suppression of food intake that was found within the whole dose range and all three formulations of CCK-8 tested (Table 12). These results demonstrate that oral administration of CCK8 using the formulations described herein significantly inhibited food intake, suggesting that the formulations facilitate permeation of the active form of CCK-8 through the GI wall.

TABLE 12

| | | | T-test with no |
|---|---|---|---|
| | Median (ml) | Average (ml) | treatment/formulation |
| No treatment | 7.71 | 7.26 ± 1.04 | |
| Formulation | 9.20 | 9.84 ± 1.61 | 0.176 (with no |
| alone (Placebo) | | | treatment) |
| CCK8 in | 7.90 | 6.80 ± 0.99 | 0.7 (with no |
| saline (0.165 mg | | | treatment) |
| per rat) | | | |
| Formulation R | 1.45 | 1.88 ± 0.5 | 0.0012 (with no |
| 0.165 mg per rat | | | treatment) |
| Formulation S | 4.10 | 4.66 ± 1.04 | 0.01 (with no |
| 0.09 mg per rat | | | treatment) |
| Formulation T | 2.25 | 4.21 ± 1.39 | 0.01 (with no |
| 0.09 mg per rat | | | treatment) |
| Formulation R | 3.70 | 5.31 ± 1.49 | 0.06 (with |
| 0.055 mg per rat | | | formulation) |
| Formulation R | 8.25 | 7.47 ± 1.19 | 0.9 (with no |
| 0.003 mg per rat | | | treatment) |

Values are median, average±standard error, and 2 samples T-test.

Having thus described several aspects of at least one embodiment, it is to be appreciated that various alterations, modifications, and improvements will readily occur to those skilled in the art. Such alterations, modifications, and improvements are intended to be part of this disclosure and are intended to be within the scope of the invention. Accordingly, the foregoing description and drawings are by way of example only, and the scope of the invention should be determined from proper construction of the appended claims, and their equivalents.

1. A pharmaceutical composition comprising a suspension which comprises an admixture of a hydrophobic medium and a solid form wherein the solid form comprises a therapeutically effective amount of CCK-8 or an analog thereof and at least one salt of a medium chain fatty acid.

2. The pharmaceutical composition of claim 1 which additionally comprises a second therapeutic agent.

3. The pharmaceutical composition of claim 2 which additionally comprises a third therapeutic agent

4. The pharmaceutical composition of claim 1 which comprises an additional constituent selected from the group consisting of a matrix forming polymer and a sugar.

5. The pharmaceutical composition of claim 1, wherein the solid form comprises a particle.

6. The pharmaceutical composition of claim 5, wherein the particle is produced by lyophilization or by granulation or by spray-drying.

7. The pharmaceutical composition of claim 1, wherein the water content in the pharmaceutical composition is lower than about 6% by weight preferably lower than about 2% by weight.

8. The pharmaceutical composition of claim 1, wherein the water content in the solid form is lower than about 6% by weight, preferably lower than 2% by weight.

9. The pharmaceutical composition of claim 1, wherein the medium chain fatty acid salt has a chain length from about 6 to about 14 carbon atoms.

10. The pharmaceutical composition of claim 6 wherein the medium chain fatty acid salt is sodium hexanoate, sodium heptanoate, sodium octanoate, sodium nonanoate, sodium decanoate, sodium undecanoate, sodium dodecanoate, sodium tridecanoate or sodium tetradecanoate, or a corresponding potassium or lithium or ammonium salt or a combination thereof.

11. The pharmaceutical composition of claim 10, wherein the fatty acid salt is sodium octanoate.

12. The pharmaceutical composition of claim 1, wherein the medium chain fatty acid salt is present in the composition at an amount of 11% to 40% by weight preferably 12% to 18% by weight, most preferably aboutl5% by weight.

13. The pharmaceutical composition of claim 1, wherein the medium chain fatty acid salt is present in the solid form at an amount of 50% to 90% by weight preferably at an amount of 70% to 80% by weight.

14. The pharmaceutical composition of claim 4, wherein the matrix forming polymer is selected from the group consisting of polyvinylpyrrolidone (PVP), cross-linked PVP, ionic polysaccharides, neutral polysaccharides, linear polyacrylic acid polymers including polymethacrylic acid polymers, cross-linked polyacrylic acid polymers, amino-polysaccharides, S-containing polymers, and high molecular weight linear and bridged organic alcohols.

15. The pharmaceutical composition of claim 4, wherein the matrix forming polymer is present in the composition at an amount of about 0.5% to 15% by weight, preferably about 1% to 10% by weight.

16. The pharmaceutical composition of claim 4 wherein the matrix forming polymer is polyvinylpyrrolidone.

17. The pharmaceutical composition of claim 16, wherein polyvinylpyrrolidone is PVP-12 and is present in the composition at an amount of about 2% to about 20% by weight, preferably at an amount of about 3% to about 18% by weight, more preferably at an amount of about 5% to about 15% by weight, most preferably at an amount of about 10% by weight.

18. The pharmaceutical composition of claim 15, wherein the cross-linked acrylic acid polymer is a sugar-cross-linked polymer, preferably a Carbopol polymer or a polyvinyl alcohol.

19. The composition of claim 18, wherein the Carbopol polymer is preferably Carbopol 934P and is present in the composition at an amount of about 0.1% to about 10% by weight, preferably at an amount of about 0.5% to about 5% by weight, most preferably at an amount of about 1% by weight.

20. The pharmaceutical composition of claim 19 which additionally comprises a surfactant.

21. The pharmaceutical composition of claim 20 wherein the surfactant comprises an ionic surfactant or a non-ionic surfactant or a combination thereof.

22. The pharmaceutical composition of claim 21 where the surfactant is lecithin or a bile salt or a detergent or a combination thereof.

23. The pharmaceutical composition of claim 19 wherein the surfactant is a monoglyceride, a cremophore, a polyethylene glycol fatty alcohol ether, a sorbitan fatty acid ester, a polyoxyethylene sorbitan fatty acid ester, Solutol HS15 (polyoxyethylene esters of 12-hydroxystearic acid), an alkyl-saccharide (e.g. octyl glycoside, tetra decyl maltoside) or a poloxamer or a combination thereof.

24. The pharmaceutical composition of claim 21 wherein the monoglyceride is glyceryl monocaprylate, glyceryl monoocatnoate, glyceryl monodecanoate, glyceryl monolaurate, glyceryl monomyristate, glyceryl monopalmitate or glyceryl monooleate or glyceryl monostearate or a combination thereof or wherein.
the sorbitan fatty acid ester comprises sorbitan monolaurate, sorbitan monooleate or sorbitan monopalmitate or a combination thereof or wherein the polyoxyethylene sorbitan fatty acid ester comprises polyoxyethylene sorbitan monooleate (Tween 80), polyoxyethylene sorbitan monostearate or polyoxyethylene sorbitan monopalmitate or a combination thereof.

25. The pharmaceutical composition of claim 18, wherein the surfactant is in the solid form.

26. The pharmaceutical composition of claim 18, wherein the surfactant is in the hydrophobic medium.

27. The pharmaceutical composition of claim 18, wherein the surfactant is in both the solid form and the hydrophobic medium.

28. The pharmaceutical composition of claim 25 wherein the surfactant is lecithin or a bile salt or a detergent or a combination thereof.

29. The pharmaceutical composition of claim 28, wherein the bile salt is sodium taurocholate, sodium deoxycholate, sodium glycocholate, sodium chenodeoxycolate, sodium cholate, sodium lithocholate, in particular sodium taurocholate.

30. The pharmaceutical composition of claim 1, wherein the hydrophobic medium comprises castor oil or glyceryl tricaprylate or glyceryl tributyrate or glyceryl monocaprylate or octanoic acid or a combination thereof.

31. The pharmaceutical composition of claim 30, wherein the main component by weight of the hydrophobic medium is castor oil or glyceryl tricaprylate or glyceryl monocaprylate or octanoic acid.

32. The pharmaceutical composition of claim 1 wherein the main component of the hydrophobic medium consists essentially of castor oil or glyceryl tricaprylate or glyceryl monocaprylate or octanoic acid.

33. The pharmaceutical composition of claim 1 wherein the hydrophobic medium comprises an aliphatic, olefinic, cyclic or aromatic compound, preferably an aliphatic compound, or a combination thereof.

34. The pharmaceutical composition of claim 1 wherein the hydrophobic medium comprises a mineral oil, a paraffin, a fatty acid such as octanoic acid, a monoglyceride, a diglyceride, a triglyceride, an ether or an ester, or a combination thereof.

35. The pharmaceutical composition of claim 24, wherein the ester in the hydrophobic medium is a low molecular weight ester, preferably ethyl isovalerate or butyl acetate.

36. The pharmaceutical composition of claim 24, wherein the triglyceride is a long chain triglyceride, a medium chain triglyceride or a short chain triglyceride or a combination thereof.

37. The pharmaceutical composition of claim 26, wherein the long chain triglyceride is castor oil.

38. The pharmaceutical composition of claim 26, wherein the short chain triglyceride is glyceryl tributyrate and the medium chain triglyceride is glyceryl tricaprylate or coconut oil.

39. The pharmaceutical composition of claim 1, wherein the composition consists essentially of cholecystokinin-8, a medium chain fatty acid salt, a matrix forming polymer and a hydrophobic medium.

40. The pharmaceutical composition of claim 1, wherein the hydrophobic medium consists essentially of glyceryl tricaprylate or glyceryl monocaprylate or a combination thereof.

41. The pharmaceutical composition of claim 1 which additionally comprises a stabilizer.

42. A pharmaceutical composition comprising a suspension which consists essentially of an admixture of a hydrophobic medium and a solid form, wherein the solid form comprises a therapeutically effective amount of CCK-8 or an analog thereof, at least one salt of a medium chain fatty acid and a matrix forming polymer wherein the matrix forming polymer is selected from the group comprising cross-linked acrylic acid polymer, polyvinyl alcohol polymer of molecular weight 10000-70000 Da, hyaluronic acid and salts thereof, PVP and cross-linked PVP.

43. The pharmaceutical composition of claim 42 which additionally comprises a second therapeutic agent.

44. The pharmaceutical composition of claim 43 which additionally comprises a third therapeutic agent.

45. The pharmaceutical composition of claim 42 wherein the matrix forming polymer is a carbomer, the medium chain fatty acid is sodium octanoate, and the hydrophobic medium comprises glyceryl tricaprylate and one or more surfactants.

46. The pharmaceutical composition of claim 45, wherein the carbomer is present at 0.1-3, preferably 1% by weight, the sodium octanoate is present at 10% or more by weight preferably 15%, and the surfactant is preferably present at about 6% by weight and is preferably lecithin or glyceryl monocaprylate or Tween 80 or a combination thereof.

47. The pharmaceutical composition of claim 42, wherein the matrix forming polymer is PVP, preferably PVP-12, the medium chain fatty acid is sodium octanoate, and the hydrophobic medium comprises glyceryl tricaprylate and surfactants, and optionally a stabilizer.

48. A pharmaceutical composition comprising a suspension which comprises an admixture of a hydrophobic medium and a solid form wherein the solid form comprises a therapeutically effective amount of CCK-8 or an analog thereof; sodium octanoate; and a matrix forming polymer, and which optionally comprises a second therapeutic agent and optionally a third therapeutic agent.

49. The pharmaceutical composition of claim 48 which additionally contains one or more surfactants.

50. The pharmaceutical composition of claim 48 which additionally contains a stabilizer and/or a peptidase inhibitor.

51. The pharmaceutical composition of claim 2, wherein the second or third therapeutic agent is selected from the group consisting of anti-obesity or appetite suppressant drugs.

52. The pharmaceutical composition of claim 51 wherein the anti-obesity or appetite suppressant drug is selected from the group consisting of orlistat, sibutramine, phendimetrazine tartrate, methamphetamine, phentermine, Adipex-P™, oxyntomodulin, an oxyntomodulin analog, PYY, PYY analog, GLP-1 and a GLP-1 analog.

53. An oral dosage form comprising the pharmaceutical composition of claim 1.

54. The oral dosage form of claim 52 which is additionally enteric-coated.

55. A rectal dosage form comprising the composition of claim 1.

56. A kit comprising instructions and the dosage form of claim 53.

57. A capsule containing the pharmaceutical composition of claim 1.

58. The capsule of claim 57, wherein the capsule is a hard gel or a soft gel capsule.

59. The capsule of claim 57, wherein the capsule is enteric-coated.

60. A method of treatment of a subject suffering from overweight comprising administering orally to the subject a therapeutically effective amount of CCK-8 sufficient to produce weight loss.

61. The method of treatment of claim 60, wherein the weight loss is accompanied by reduction in liver size.

62. The method of treatment of claim 60, wherein the overweight subject is obese.

63. The method of treatment of claim 60, wherein the administration is prior to surgery.

64. The method of treatment of claim 63, wherein the surgery is bariatric surgery.

65. The method of treatment of claim 63, wherein the administration period is 6 months or less prior to surgery.

66. The method of treatment of claim 65, wherein the administration period is 2-8 weeks prior to surgery.

67. The method of treatment of claim 60, wherein the treatment is for acute use.

68. The method of treatment of claim 60, wherein the treatment is for chronic use

69. The method of treatment of claim 60, wherein the administration is prior to a meal.

70. The method of treatment of claim 60, wherein the administration is one, two, three, four or five times per day.

71. The method of treatment of claim 60, wherein the CCK-8 administration is within an enteric coated capsule or tablet.

72. The method of treatment of claim 60, wherein the CCK-8 administered is the pharmaceutical composition of claim 1.

73. A method of treatment of a subject desirous of weight control comprising administering orally to the subject a therapeutically effective amount of CCK-8 sufficient to achieve weight control by the subject.

74. (canceled)

75. A method of treatment of claim 63, wherein the liver size is measured before commencing of CCK-8 treatment and again just prior to surgery.

76. A method of treatment of claim 63, wherein the liver size is measured before, during and after a period of administration of CCK-8.

77. A method of treating a subject suffering from bulimia nervosa or binge eating disorder, which comprises administering to the subject an oral composition of cholecystokinin-8 in an amount sufficient to treat the condition.

78. A method of stimulating gallbladder contraction in a subject which comprises administering to the subject an oral composition of CCK-8 in an amount sufficient to stimulate gallbladder contraction.

79. A process for producing a pharmaceutical composition which comprises preparing a water-soluble composition comprising a therapeutically effective amount of CCK-8 and optionally a second and optionally a third therapeutic agent, a medium chain fatty acid salt and a matrix forming polymer, drying the water soluble composition to obtain a solid powder, and suspending the solid powder in a hydrophobic medium, to produce a suspension containing in solid form the therapeutic agent, the medium chain fatty acid salt and the matrix forming polymer, thereby producing the pharmaceutical composition.

80. A process for producing a pharmaceutical composition which comprises providing a solid powder comprising a therapeutically effective amount of CCK-8 and optionally a second and optionally a third therapeutic agent, a medium chain fatty acid salt and a matrix forming polymer, and suspending the solid powder in a hydrophobic medium, to produce a suspension containing in solid form the therapeutic agent, the medium chain fatty acid salt and the matrix forming polymer, thereby producing the pharmaceutical composition.

81. A pharmaceutical composition comprising a suspension which comprises an admixture of a hydrophobic medium and a solid form wherein the solid form comprises a therapeutically effective amount of a therapeutic agent, at least one salt of a medium chain fatty acid, a bile salt and an additional constituent selected from the group consisting of a matrix forming polymer and a sugar.
